(19) United States
(12) Patent Application Publication
(10) Pub. No.: US 2025/0073163 A1
(43) Pub. Date: Mar. 6, 2025

(54) METHOD FOR TREATING INFECTIOUS DISEASE

(71) Applicant: Suntec Medical, Inc., Walnut, CA (US)

(72) Inventors: Chun-Ting Cheng, Taipei (TW); Yuan-Chung Tsai, Taipei (TW); Pauline Ying Lau, Walnut, CA (US)

(21) Appl. No.: 18/939,350

(22) Filed: Nov. 6, 2024

Related U.S. Application Data
(63) Continuation of application No. PCT/US2023/021585, filed on May 9, 2023.
(60) Provisional application No. 63/341,547, filed on May 13, 2022.

(51) Int. Cl. A61K 9/107; A61K 38/20; A61K 38/21

(52) U.S. Cl. CPC A61K 9/1075; A61K 38/2013; A61K 38/204; A61K 38/208; A61K 38/2086; A61K 38/212; A61K 38/217

(57) ABSTRACT

The present invention provides a method of treating an infectious disease. The method comprises the step of administering to a subject in need thereof an effective amount of (i) a polymer-flavonoid conjugate, (ii) a flavonoid oligomer, or (iii) micelles having a shell formed by one or more polymer-flavonoid conjugates or one or more flavonoid oligomers, or the combination thereof, and having an agent encapsulated within the shell. The present method is effective to treat viral infection, e.g., severe acute respiratory syndrome coronavirus (SARS-CoV), enterovirus virus, HIV, hepatitis B virus, MERS-CoV, influenza virus, Dengue virus, respiratory syncytial virus, hepatitis C virus, monkeypox virus, human papillomavirus, methicillin-resistant Staphylococcus aureus, Pseudomonas, tuberculosis, Bacillus anthracis, Tetani bacterium, Streptococcus pneumoniae, meningococcus, Escherichia coli, Legionella, Neisseria gonorrhea, Neisseria meningitidis, and Salmonella.

### CROSS-REFERENCE TO RELATED APPLICATION(S)

This application is a continuation of PCT/US2023/021585, filed May 9, 2023; which claims the benefit of U.S. Provisional Application No. 63/341,547, filed May 13, 2022. The contents of the above-identified applications are incorporated herein by reference in their entireties.

### FIELD OF THE INVENTION

The present invention provides a method of treating infectious diseases such as bacterial infection and viral infection. The method comprises the step of administering to a subject in need thereof an effective amount of (i) a polymer-flavonoid conjugate, (ii) a flavonoid oligomer, or (iii) micelles having a shell formed by one or more polymer-flavonoid conjugates or one or more flavonoid oligomers, or the combination thereof, and having an active agent encapsulated within the shell.

### BACKGROUND OF THE INVENTION

### Infectious Diseases

Infectious diseases are disorders caused by pathogenic organisms such as bacteria, viruses, fungi, or parasites. An infectious disease is the invasion of body tissues by disease-causing agents, their multiplication, and the reaction of host tissues to the infectious agents and the toxins they produce.

The bacterial infection and viral infections constitute most of the infectious diseases. Hosts can fight infections using their immune system. Mammalian hosts react to infections with an innate response, often involving inflammation, followed by an adaptive response. The typical signs and symptoms of an infection are signs of host inflammation caused by these pathogens, such as fatigue, loss of appetite, weight loss, fevers, night sweats, chills, aches and pains, skin rashes, coughing or a runny nose.

### Bacterial Infection

A bacterial infection is a proliferation of a harmful strain of bacteria on or inside the body. Bacterial infection occurs when bacteria enter the body, increase in number, and cause a reaction in the body. Bacteria can enter the body through an opening in the skin, through the airway, the digestive system, and cause infections. Bacteria can infect any area of the body. Pneumonia, meningitis, and food poisoning are just a few illnesses that may be caused by harmful bacteria. Bacteria come in three basic shapes: rod-shaped (bacilli), spherical (cocci) or helical (spirilla). Bacteria may also be classified as gram-positive or gram-negative. Gram-positive bacteria have a thick cell wall while gram-negative bacteria do not. Gram staining, bacterial culture with antibiotic sensitivity determination, and other tests like genetic analysis are used to identify bacterial strains and help determine the appropriate course of treatment.

Bacteria cause disease by secreting or excreting toxins (as in botulism), by producing toxins internally, which are released when the bacteria disintegrate (as in typhoid) or by inducing sensitivity to their antigenic properties (as in tuberculosis).

Some serious bacterial diseases include cholera, diphtheria, bacterial meningitis, tetanus, lyme disease, gonorrhea, syphilis, tuberculosis, anthrax, tetanus, keptospirosis, pneumonia, botulism, Pseudomonas infection, MRSA infection, E. coli infection and bubonic plague.

### Viral Infection

A viral disease (or viral infection) occurs when an organism's body is invaded by pathogenic viruses, and infectious virus particles (virions) attach to and enter susceptible cells. These viruses include but not limited to adenovirus, coxsackievirus, cytomegalovirus, Epstein-Barr virus, hepatitis A virus, hepatitis B virus, hepatitis C virus, herpes simplex virus, type 1, herpes simplex virus, type 2, HIV, human coronavirus 229E (HCoV-229E), human coronavirus HKU1 (HCoV-HKU1), human coronavirus NL63 (HCoV-NL63), human coronavirus OC43 (HCoV-OC43), human herpesvirus, human papillomavirus, influenza virus, measles virus, middle east respiratory syndrome-related coronavirus (MERS-CoV), mumps virus, parainfluenza virus, poliovirus, rabies virus, respiratory syncytial virus, rubella virus, severe acute respiratory syndrome coronavirus (SARS-CoV), severe acute respiratory syndrome coronavirus 2 (SARS-CoV-2), varicella-zoster virus

A viral infection is a proliferation of a harmful virus inside a host. Viruses cannot reproduce without the assistance of a host. Viruses infect a host by introducing their genetic material into the cells and using the cell's internal machinery to make more virus particles.

### Antibiotic Treatment

Bacterial infection has been treated by antibiotics. But the pipeline of new drugs is drying up. For example, nearly 40 years elapsed between introduction of the two newest molecular classes of antibiotics: fluoroquinolones (such as Cipro) in 1962 and the oxazolidinones (such as Zyvox) in 2000. Major pharmaceutical companies have limited interest in dedicating resources to the antibiotics market because these short-course drugs are not as profitable as drugs that treat chronic conditions and lifestyle-related ailments, such as high blood pressure or high cholesterol. Antibiotic research and development are also expensive, risky, and time consuming. Return on that investment can be unpredictable, considering that resistance to antibiotics develops over time, eventually making them less effective.

### Flavonoids

Flavonoids have the general structure of a 15-carbon skeleton, which consists of two phenyl rings (A and B) and a heterocyclic ring (C, the ring containing the embedded oxygen).

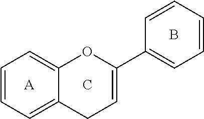

This carbon structure can be abbreviated C6-C3-C6. According to the IUPAC nomenclature, flavonoids can be classified into:
flavonoids or bioflavonoids
isoflavonoids, derived from 3-phenylchromen-4-one (3-phenyl-1,4-benzopyrone) structure
neoflavonoids, derived from 4-phenylcoumarine (4-phenyl-1,2-benzopyrone) structure

### BRIEF DESCRIPTION OF THE DRAWINGS

FIG. 1 illustrates one embodiment of a MINC (Multi-pathway Immune-modulating Nanocomplex Combination therapy)-drug, which is a micelle having a polymer-flavonoid conjugate, for example, a PEG-EGCG conjugate, in a shell and having a drug encapsulated.

FIG. 2 illustrates another embodiment of a MINC-agent, which is a micelle comprises a polymer-flavonoid conjugate, e.g., a PEG-EGCG conjugate in an outer shell and a flavonoid oligomer, for example, oligomeric EGCG (OEGCG), in an inner shell, and having a drug encapsulated.

FIG. 3 shows that OEGCG reduced the colony formation of methicillin resistant Staphylococcus aureus (MRSA).

FIG. 4 shows that OEGCG reduced the colony formation of Pseudomonas aeruginosa,

FIG. 5 shows that OEGCG and PEG-EGCG suppressed SARS-CoV-2 infection.

FIG. 6 shows that OEGCG suppressed EV D68 infection.

FIG. 7 shows that different polymer-flavonoid conjugate and different flavonoid oligomers all successfully generated MINC-anti-HER2 micelles with particle size around 100 nm.

FIG. 8 demonstrates that different polymers in polymer-flavonoid conjugates successfully generated MINC-BSA.

FIG. 9 shows a successful formulation of MINC-IFN-α.

FIG. 10 shows a successful formulation of MINC-IFN-γ.

FIG. 11 shows a successful formulation of MINC-IL-12.

FIG. 12 shows a successful formulation of MINC-IL-2.

FIG. 13 shows a successful formulation of MINC-IL-6.

FIG. 14 shows a successful formulation of MINC-IL-15.

FIG. 15 shows a successful formulation of MINC-IL-21.

### DETAILED DESCRIPTION OF THE INVENTION

### Definitions

The term “epigallocatechin gallate” refers to an ester of epigallocatechin and gallic acid, and is used interchangeably with “epigallocatechin-3-gallate” or EGCG.

The term “oligomeric EGCG” (OEGCG) refers to 3-20 monomers of EGCG that are covalently linked. OEGCG preferably contains 4 to 12 monomers of EGCG.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate.

The term “nucleoside analogue” refers to a nucleoside where a natural base (A, T, G, C, or U) or a natural ribose/deoxyribose is modified. Nucleoside analogues can be used to prevent viral replication in infected cells.

The term “nucleotide analogue” refers to a nucleotide where a natural base (A, T, G, C, or U), a natural ribose/deoxyribose or phosphate is modified. Nucleotide analogues can be used to prevent viral replication in infected cells. Remdesivir for example is a nucleotide analogue.

The present invention provides a method of treating infectious diseases. The method comprises the step of administering to a subject in need thereof an effective amount of (i) a polymer-flavonoid conjugate, (ii) a flavonoid oligomer, or (iii) micelles having a shell formed by one or more polymer-flavonoid conjugates or one or more flavonoid oligomers, or the combination thereof, and having an agent encapsulated within the shell.

### Flavonoid

Flavonoids suitable for the present invention have the general structure of Formula I:

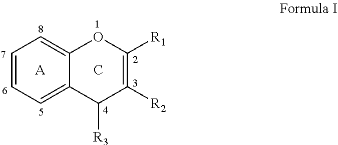

wherein:
R₁ is H, or phenyl;
R₂ is H, OH, Gallate, or phenyl; wherein the phenyl is optionally substituted by one or more (e.g., 2-3) hydroxyl;
R₃ is H, OH, or ═O (oxo); or
R₁ and R₂ together form a close-looped ring structure; or
R₂ and R₃ together form close-looped ring structure.

The 2, 3, 4, 5, 6, 7, or 8 position of Formula I, can be linked to a group containing hydrocarbon, halogen, oxygen, nitrogen, sulfur, phosphorus, boron or metals.

Examples of flavonoids of Formula I include:

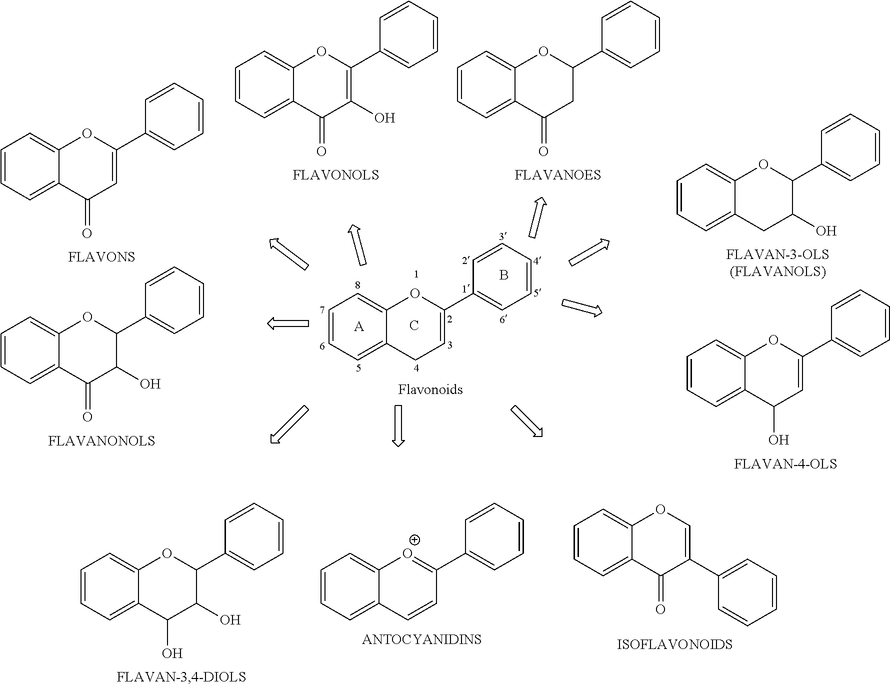

Preferred flavonoid compounds of Formula I include:
EGCG (CAS #989-51-5), EC (CAS #490-46-0), EGC (CAS #970-74-1) or ECG (CAS #1257-08-5)

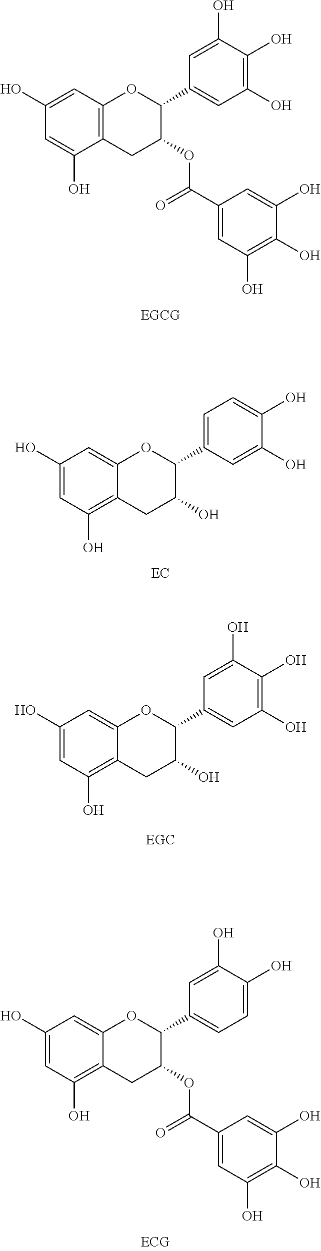 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]

### Polymer-Flavonoid Conjugate

A polymer-flavonoid conjugate, as used herein throughout the application, refers to a conjugate of a hydrophilic polymer and the flavonoid compound of Formula I.

A hydrophilic polymer refers to a polymer that is soluble in polar solvents and can form hydrogen bonds. Hydrophilic polymers suitable for the present polymer-flavonoid conjugates include, but not limited to: poly(ethylene glycol) (PEG), aldehyde-derivatized hyaluronic acid, hyaluronic acid, dextran, diethylacetal conjugate (e.g. diethylacetal PEG), D-alpha-tocopheryl polyethylene glycol succinate, aldehyde-derivatized hyaluronic acid-tyramine, hyaluronic acid-aminoacetylaldehyde diethylacetal conjugate-tyramine, cyclotriphosphazene core phenoxymethyl(methylhydrazono)dendrimer or thiophosphoryl core phenoxymethyl(methylhydrazono)dendrimer, acrylamides, oxazolines, imines, acrylic acids, methacrylates, diols, oxiranes, alcohols, amines, anhydrides, esters, lactones, terephthalate, amides and ethers polyacrylamide, poloxamers, poly(N-isopropylacrylamide), poly(oxazoline), polyethylenimine, poly(acrylic acid), polymethacrylate, poly(ethylene glycol), poly(ethylene oxide), poly(vinylalcohol), poly(vinylpyrrolidinone), polyethers, poly(allylamine), polyanhydrides, poly(β-amino ester), poly(butylene succinate), polycaprolactone, polycarbonate, polydioxanone, poly(glycerol), polyglycolic acid, poly(3-hydroxypropionic acid), poly(2-hydroxyethyl methacrylate), poly(N-(2 hydroxypropyl)methacrylamide), polylactic acid, poly(lactic-co-glycolic acid), poly(ortho esters), poly(2 oxazoline), poly(sebacic acid), poly(terephthalate-co-phosphate), povidone and copolymers.

Preferred hydrophilic polymers include poly(ethylene glycol), hyaluronic acid, dextran, polyethylenimine, poloxamers, povidone, D-alpha-tocopheryl and polyethylene glycol succinate. The molecular weight of the hydrophilic polymer in the polymer-flavonoid conjugate is in general 1K-100K, preferably 2K-40K, 2K-50K, 2K-80K, 3K-80K, or 5K-40K Daltons.

In one embodiment, the polymer contains an aldehyde group which is conjugated to the 5, 6, 7, or 8 position (preferably 6 or 8 position) of the A ring of the flavonoid compound. In another embodiment, the polymer contains a thiol group which is conjugated to R₁ or R₂ of the B-ring of a flavonoid (when R₁ or R₂ is —OH).

In one embodiment, the polymer-flavonoid conjugate is PEG-EGCG, which is PEG conjugated to one or two molecules of epigallocatechin gallate (EGCG). PEG-EGCG, for example, can be prepared by conjugating aldehyde-terminated PEG to EGCG by attachment of the PEG via reaction of the free aldehyde group with the 5, 6, 7, or 8 position (preferably 6 or 8 position) of Formula I. See WO2006/124000 and WO2009/054813. PEG-EGCG can also be prepared by conjugating thio-terminated PEG to EGCG by attachment of the PEG via reaction of the free thio group with the R1 or R2 of Formula I, wherein, R1 or R2 is a phenyl group. See WO2015/171079.

### Flavonoid Oligomer

A flavonoid oligomer is a conjugate of one flavonoid with one or more flavonoids. The flavonoid oligomer can contain the same flavonoid (a homo oligomer) or different flavonoids (a hetero oligomer). Flavonoid oligomers useful for the present invention in general have 2-50 or 2-20, preferably 4-12 flavonoids of one or mixed types.

In some embodiment, a flavonoid oligomer is oligomeric EGC (OEGCG), oligomer EC (OEC), oligomer EGC (OEGC) or oligomer ECG (OECG). OEGCG refers to 3-20 monomers of EGCG that are covalently linked. OEGCG, for example, can be synthesized at 5, 6, 7, or 8 position (preferably 6 or 8 position) of the A ring according to WO2006/124000.

Because A-ring is present in all the flavonoids according to Formula 1, other oligomeric flavonoids can be made similarly according to WO2006/124000. For example, OEC, OEGC, and OECG can also be made according to WO2006/124000.

### MINC-Drug

MINC (Multi-pathway Immune-modulating Nanocomplex Combination therapy) is a platform technology, utilizing the bioactivity of polymer-flavonoid conjugates or flavonoid oligomers that form micelles in a solution. MINC platform can encapsulate additional agents such as drugs to form a nanoparticle composition for a combination therapy.

MINC-drug is a micelle with a shell formed by one or more polymer-flavonoid conjugates or one or more flavonoid oligomers, or the combination thereof, and has a drug encapsulated within the shell. The drug, as used herein, referred to a molecule that have a therapeutic activity to treat infection by bacteria or viruses, including but not limited to, anti-inflammatory agents, nucleotide/nucleoside analogues and agents to boost host immune defensive responses. Host immune defensive agents are mostly anti-inflammatory cytokines, IL-1, IL-2, IL-3, IL-4, IL-5, IL-6, IL-7, IL-8, IL-10, IL-12, IL-13, IL-15, IL-17, IL-18, IFN-α, IFN-β, IFN-γ, TNF-α, G-CSF, GM-CSF or M-CSF.

In one embodiment, MINC-drug is a micelle comprises a polymer-flavonoid conjugate, e,g., a PEG-EGCG conjugate, in a shell and with an agent encapsulated (see FIG. 1).

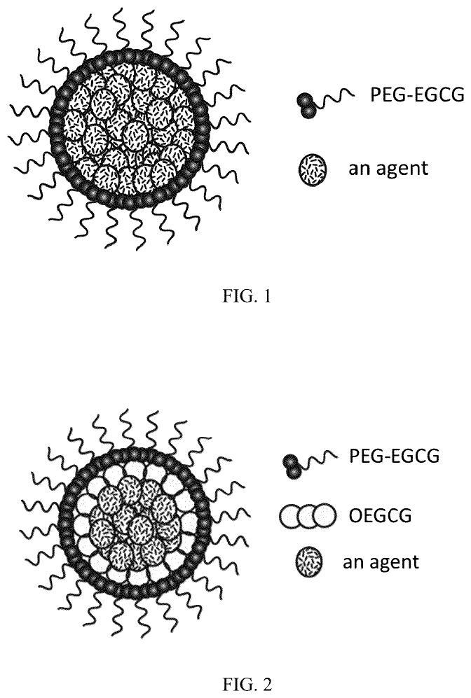

In another embodiment, MINC-drug is a micelle comprises a polymer-flavonoid conjugate, e.g., a PEG-EGCG conjugate in an outer core and a flavonoid oligomer, e.g;, oligomeric EGCG (OEGCG), in an inner core, with an agent encapsulated (see FIG. 2).

“Polymer-flavonoid conjugate” or “Flavonoid oligomers” can be used for treating infectious diseases including viral or bacterial infections by the mechanisms, including but not limited, to suppressing viral or bacterial replication; killing viruses or bacteria; preventing viral or bacterial from entering/infecting their targeted cells/tissues/organs; preserving the functions/activities of the infected cells/tissues/organs; regulating host immunity to kill infectious microbes.

The MINC-drug composition comprises two or more components that have therapeutic activities, which are complementary in function to form a multiple targeted combination therapy by its backbone components (polymer-flavonoid conjugates or flavonoid oligomers), and the encapsulated drug (the Agent). The MINC-drug is stable in a hydrophilic environment, such as blood circulation.

In one embodiment, the agent is a cytokine including but not limited to IL-1, IL-2, IL-3, IL-4, IL-5, IL-6, IL-7, IL-8, IL-10, IL-12, IL-13, IL-15, IL-17, IL-18, IFN-α, IFN-β, IFN-γ, TNF-α, G-CSF, GM-CSF or M-CSF. These cytokines are for cross-disease-type treatment; the purpose is to induce host immune response to fight infectious microbes, not restricted to a specific type of infectious disease. These cytokines are suitable for treating virus infection including adenovirus, coxsackievirus, cytomegalovirus, enterovirus, Epstein-Barr virus, hepatitis A virus, hepatitis B virus, hepatitis C virus, herpes simplex virus, type 1, herpes simplex virus, type 2, HIV, human coronavirus 229E (HCoV-229E), human coronavirus HKU1 (HCoV-HKU1), human coronavirus NL63 (HCoV-NL63), human coronavirus OC43 (HCoV-OC43), human herpesvirus, human papillomavirus, influenza virus, measles virus, middle east respiratory syndrome-related coronavirus (MERS-CoV), mumps virus, parainfluenza virus, poliovirus, rabies virus, respiratory syncytial virus, rubella virus, rotavirus, Severe acute respiratory syndrome coronavirus (SARS-CoV), severe acute respiratory syndrome coronavirus 2 (SARS-CoV-2) or varicella-zoster virus. The cytokines are suitable for treating bacterial infection including but not limited to Mycobacterium tuberculosis, Clostridium tetani, Salmonella typhi, Corynebacterium diphtheria, Treponema pallidum, Mycobacterium lepromatosis or methicillin-resistant Staphylococcus aureus.

In one embodiment, the agent is an antiviral antibody including but not limited to anti-SARS-CoV-2-RBD, anti-SARS-CoV-2-SP, anti-SARS-CoV-2-SD1/SD2, anti-SARS-CoV-2-S2, Bamlanivimab, Etesevimab, Casirivimab, Imdevimab, Cilgavimab, Tixagevimab, Sotrovimab or Regdanvimab for treating SARS-CoV-2 infection, which causes COVID-19.

In one embodiment, the agent is an antiviral antibody, including but not limited to anti-HIV-gp120, VRC01, 10-1074, anti-HIV-Env, 3BNC117, anti-HIV-gp41, 4E10, 2F5, 2G12, anti-CCR5, PRO 140, anti-CD4, Ibalizumab or Leronlimab for treating HIV (human immunodeficiency virus) infection, which causes AIDS.

In one embodiment, the agent is an antiviral antibody including but not limited to anti-HPV-E6 or anti-HPV-E7 for treating HPV (Human papillomavirus) infection, which causes cervical cancer.

In one embodiment, the agent is an antiviral antibody including but not limited to anti-CMV-gB, CSJ148, TCN-202, anti-CMV-gH, LPJ539, Anti-CMV-gH/gH, MCMV5322A, Sevirumab or MCMV3068A for treating CMV (Cytomegalovirus) infection.

In one embodiment, the agent is an antiviral antibody including but not limited to anti-influenza A hemagglutinins, MHAA4549A, VIS410, CR6261, CR8020 or TCN-032 for treating influenza virus infection.

In one embodiment, the agent is an antiviral antibody including but not limited to anti-RSV-glycoprotein F, Palivizumab, REGN2222, Motavizumab, MEDI8897, ALX-0171 or Nirsevimab for treating RSV (Respiratory syncytial virus) infection.

In one embodiment, the agent is an antiviral antibody including but not limited to ZMapp, atoltivimab, maftivimab or odesivimab for treating Ebola virus.

In one embodiment, the agent is an antiviral antibody including but not limited to CR57, CR4098, CL184 or RAB-1 for treating Rabies virus infection.

In one embodiment, the agent is an antiviral nucleoside analogue including but not restricted to Ribavirin, Favipiravir, Lopinavir, Ritonavir or Nafamostat for treating SARS-CoV-2 infection.

In one embodiment, the agent is an antibacterial antibody, including but not limited to AR301, MEDI4893, 514G3 or ARN-100 for treating Staphylococcus aureus infection.

In one embodiment, the agent is an antibacterial antibody, including but not limited to MEDI3902 or AR101 for treating Pseudomonas aeruginosa infection.

In one embodiment, the agent is an antibacterial antibody, including but not limited to PolyCAb or Cd-ISTAb for treating Clostridium difficile infection.

In one embodiment, the agent is an antibacterial antibody, including but not limited to AR401-mAb or VXD-003 for treating Acinetobacter baumannii infection.

In one embodiment, the agent is an antibacterial antibody, including but not limited to ASN-4 for treating Escherichia coli (ST131) infection.

In one embodiment, the agent is an antibacterial antibody, including but not limited to ASN-5 for treating Klebsiella pneumoniae infection.

In one embodiment, the agent is an antiviral nucleotide analogue including but not restricted to Remdesivir for treating SARS-CoV-2 infection.

In one embodiment, the agent is an antiviral nucleoside analogue including but not restricted to Abacavir, Didanosine, Emtricitabine, Stavudine or Zidovudine for treating HIV (human immunodeficiency virus) infection.

In one embodiment, the agent is an antiviral nucleotide analogue including but not restricted to Tenofovir, Tenofovir diphosphate, d4TMP, d4TTP, AzTMP or AzTTP for treating HIV (human immunodeficiency virus) infection.

In one embodiment, the agent is an antiviral nucleoside analogue such as Acyclovir, Famciclovir or Valacyclovir for treating HSV (herpes simplex virus) infection.

In one embodiment, the agent is an antiviral nucleoside analogue such as Adefovir, Emtricitabine, Entecavir, Lamivudine, Telbivudine, Tenofovir or Ribavirin for treating HBV (hepatitis B virus) infection.

In one embodiment, the agent is an antiviral nucleotide analogue such as 3TCMP, 3TCTP, Acyclovir monophosphate, Acyclovir triphosphate, Telbivudine monophosphate or Telbivudine triphosphate for treating HBV (hepatitis B virus) infection.

In one embodiment, the agent is an antiviral nucleoside analogue such as Sofosbuvir or Ribavirin for treating HCV (hepatitis C virus) infection.

In one embodiment, the agent is an antiviral nucleotide analogue such as Ribavirin monophosphate or Ribavirin triphosphate for treating HCV (hepatitis C virus) infection.

In one embodiment, the agent is an antiviral nucleoside analogue such as Cidofovir, Ganciclovir or Valganciclovir for treating CMV (cytomegalovirus) infection.

In one embodiment, the agent is an antiviral nucleoside analogue such as Famciclovir or Valacyclovir for treating VZV (varicella-zoster virus) infection.

In one embodiment, the agent is an antiviral nucleotide analogue such as Acyclovir monophosphate or Acyclovir triphosphate for treating VZV (varicella-zoster virus) infection.

In one embodiment, the agent is an antiviral nucleoside analogue such as Ribavirin, 5-azacytidine, and 5-fluorouracil, Zanamivir, Oseltamivir, Peramivir or Baloxavir for treating influenza virus infection.

In one embodiment, the agent is an antiviral nucleotide analogue such as Ribavirin monophosphate or Ribavirin triphosphate for treating influenza virus infection.

In one embodiment, the agent is IFN-α for treating viral or bacterial infection. For example, the MINC-drug is IFN-α encapsulated in the micelle formed by a polymer-flavonoid conjugate PEG-EGCG and a flavonoid oligomer OEGCG. See WO2009/054813 for the structure and formulation method.

In one embodiment, the agent is not an antibiotic.

### Pharmaceutical Compositions

The present invention uses pharmaceutical compositions comprising the polymer-flavonoid conjugate, flavonoid oligomer, or MINC-drug composition as described in the application, and optionally one or more pharmaceutically acceptable carriers. The nanoparticle composition in a pharmaceutical composition in general is about 1-90%, preferably 20-90%, or 30-80% for a tablet, powder, or parenteral formulation. The polymer-flavonoid conjugate, flavonoid oligomer, or MINC-drug composition in a pharmaceutical composition in general is 1-100%, preferably 20-100%, 50-100%, or 70-100% for a capsule formulation. The nanoparticle composition in a pharmaceutical composition in general is 1-50%, 5-50%, or 10-40% for a liquid suspension formulation.

In one embodiment, the pharmaceutical composition can be in a dosage form such as tablets, capsules, granules, fine granules, powders, suspension, patch, parenteral, injectable, or the like. The above pharmaceutical compositions can be prepared by conventional methods.

Pharmaceutically acceptable carriers, which are inactive ingredients, can be selected by those skilled in the art using conventional criteria. The pharmaceutically acceptable carriers may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate. Such pharmaceutically acceptable carriers may be preserved against bacterial contamination using well-known preservatives, which include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet, capsule, or parenteral formulation of the active compound may contain other excipients that have no bioactivity and no reaction with the active compound. Excipients of a tablet or a capsule may include fillers, binders, lubricants and glidants, disintegrators, wetting agents, and release rate modifiers. Examples of excipients of a tablet or a capsule include, but are not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone.

For example, a tablet formulation may contain inactive ingredients, such as colloidal silicon dioxide, crospovidone, hypromellose, magnesium stearate, microcrystalline cellulose, polyethylene glycol, sodium starch glycolate, and titanium dioxide. A capsule formulation may contain inactive ingredients, such as gelatin, magnesium stearate, and titanium dioxide. A powder oral formulation may contain inactive ingredients, such as silica gel, sodium benzoate, sodium citrate, sucrose, and xanthan gum.

The pharmaceutical composition can be applied by local administration and systemic administration. Local administration includes topical administration. Systemic administration includes oral, parenteral (such as intravenous, intramuscular, subcutaneous, or rectal), and other systemic routes of administration. In systemic administration, the active compound first reaches plasma and then distributes into target tissues. Parenteral administration, such as intravenous bolus injection or intravenous infusion, and oral administration are preferred routes of administration.

### Method of Treatment

The present invention is directed to a method of treating infectious diseases including viral infection and bacterial infection. The infection is caused by DNA viruses, RNA viruses, gram-positive bacteria, or gram-negative bacteria.

Suitable viral infectious diseases to be treated by the present invention include, but not limited to viral infection by adenovirus, coxsackievirus, cytomegalovirus, Epstein-Barr virus, hepatitis A virus, hepatitis B virus, hepatitis C virus, herpes simplex virus, type 1, herpes simplex virus, type 2, HIV, human coronavirus 229E (HCoV-229E), human coronavirus HKU1 (HCoV-HKU1), human coronavirus NL63 (HCoV-NL63), human coronavirus OC43 (HCoV-OC43), human herpesvirus, human papillomavirus, influenza virus, measles virus, middle east respiratory syndrome-related coronavirus (MERS-CoV), mumps virus, parainfluenza virus, poliovirus, rabies virus, respiratory syncytial virus, rubella virus, severe acute respiratory syndrome coronavirus (SARS-CoV), severe acute respiratory syndrome coronavirus 2 (SARS-CoV-2) or varicella-zoster virus.

Suitable bacterial infectious diseases to be treated by the present invention include, but not limited to bacterial infection by bacilli, cocci or spirilla bacteria caused tuberculosis, anthrax, tetanus, leptospirosis, pneumonia, cholera, botulism, Pseudomonas infection, MRSA infection, E. coli infection, meningitis, gonorrhea, bubonic plague or syphilis.

### Polymer-Flavonoid

In a first aspect of the invention, the method comprises the step of administering an effective amount of a polymer-flavonoid conjugate to a subject in need thereof. “An effective amount” as used in this application, is the amount effective to treat a disease by ameliorating the pathological condition or reducing the symptoms of the disease.

The polymer-flavonoid conjugate of the present invention has immune modulation, antiviral and antibacterial activity for infectious diseases.

In one embodiment, the flavonoid is EGCG, EC, EGC, or ECG.

In one embodiment, the polymer is a hydrophilic polymer having a molecular weight of 1,000 to 100,000 daltons, and is selected from the group consisting of: PEG, hyaluronic acid, dextran, polyethylenimine, poloxamers, povidone, D-alpha-tocopheryl, and polyethylene glycol succinate.

A preferred polymer-flavonoid conjugate is PEG-EGCG.

In one embodiment, the infection is caused by a DNA virus selected from the group consisting of: hepatitis B virus, human herpesvirus, human papillomavirus, herpes simplex virus, Epstein-Barr virus, cytomegalovirus, monkeypox virus and Varicella-zoster virus.

In one embodiment, the infection is caused by an RNA virus selected from the group consisting of: SARS-CoV-2, enterovirus virus, HIV, MERS-CoV, hepatitis C virus, hepatitis A virus, rotavirus, norovirus, influenza virus, parainfluenza virus, Dengue virus, respiratory syncytial virus, and SARS-CoV.

In one embodiment, the infection is caused by a gram-positive bacterium selected from the group consisting of: methicillin-resistant Staphylococcus aureus, tuberculosis, Bacillus anthracis, Tetani bacterium, Streptococcus pneumoniae, Clostridium botulinum, Clostridia, Mycobacterium tuberculosis, Clostridium tetani, Corynebacterium diphtheria, and Mycobacterium leprae.

In one embodiment, the infection is caused by a gram-negative bacterium selected from the group consisting of: Pseudomonas, Meningococcus, Leptospira, Neisseria gonorrhoeae, Neisseria meningitidis, Yersinia pestis, Treponema pallidum, Acinetobacter baumannii, Escherichia coli, Klebsiella pneumoniae, Mycobacterium tuberculosis, Legionella and Salmonella.

Dosing for a polymer-flavonoid, e.g., PEG-EGGC, for injection, is in general 0.1-10000 mg/kg, 0.6-1200 mg/kg (total weight of the polymer-flavonoid/subject body weight), or 1-1000 mg/kg.

### Flavonoid Oligomer

In a second aspect of the invention, the method comprises the step of administering an effective amount of a flavonoid oligomer to a subject in need thereof. The flavonoid oligomer of the present invention has immune modulation, antiviral and antibacterial activity for infectious diseases.

In one embodiment, the flavonoid is EGCG, EC, EGC, or ECG.

In one embodiment, the flavonoid oligomer comprises 4-12 flavonoids of EGCG, EC, EGC, or ECG.

A preferred flavonoid oligomer is oligomer of EGCG.

In one embodiment, the infection is caused by a DNA virus selected from the group consisting of: hepatitis B virus, human herpesvirus, human papillomavirus, herpes simplex virus, Epstein-Barr virus, cytomegalovirus, monkeypox virus and varicella-zoster virus.

In one embodiment, the infection is caused by an RNA virus selected from the group consisting of: SARS-CoV-2, enterovirus virus, HIV, MERS-CoV, hepatitis C virus, hepatitis A virus, rotavirus, norovirus, influenza virus, parainfluenza virus, Dengue virus, respiratory syncytial virus, and SARS-CoV.

In one embodiment, the infection is caused by a gram-positive bacterium selected from the group consisting of: methicillin-resistant Staphylococcus aureus, tuberculosis, Bacillus anthracis, Tetani bacterium, Streptococcus pneumoniae, Clostridium botulinum, Clostridia, Mycobacterium tuberculosis, Clostridium tetani, Corynebacterium diphtheria, and Mycobacterium leprae.

In one embodiment, the infection is caused by a gram-negative bacterium selected from the group consisting of: Pseudomonas, Meningococcus, Leptospira, Neisseria gonorrhoeae, Neisseria meningitidis, Yersinia pestis, Treponema pallidum, Acinetobacter baumannii, Escherichia coli, Klebsiella pneumoniae, Mycobacterium tuberculosis, Legionella and Salmonella.

Dosing for a flavonoid oligomer, e.g., OEGCG, for injection, is in general 0.1-1000 mg/kg, 0.1-100 mg/kg (total weight of the flavonoid oligomer/subject body weight), or 1-100 mg/kg.

### MINC-Drug

In a third aspect of the invention, the method comprises the step of administering to a subject in need thereof an effective amount of micelles having an outer shell comprising one or more polymer-flavonoid conjugates and optionally an inner shell comprising one or more flavonoid oligomer and a drug encapsulated within the shells, to treat bacterial or viral infection. In one embodiment, the shell is formed by one or more polymer-flavonoid conjugates. In one embodiment, the outer shell is formed by one or more polymer-flavonoid conjugates and the inner shell is formed by one or more flavonoid oligomer. The polymer-flavonoid conjugate or the flavonoid oligomer provides its own therapeutic effect and further delivers said agent for treating infectious diseases.

In one embodiment, the polymer is a hydrophilic polymer having a molecular weight of 1,000 to 100,000 Daltons, and is selected from the group consisting of: poly(ethylene glycol) (PEG), hyaluronic acid, dextran, polyethylenimine, poloxamers, povidone, D-alpha-tocopheryl, and polyethylene glycol succinate.

In one embodiment, the flavonoid oligomer comprises 2-20 flavonoids of EGCG, EC, EGC, or ECG.

In one embodiment, the shell is formed by PEG-EGCG.

In one embodiment, the shell is formed by PEG-EGCG and OEGCG.

In one embodiment, the drug in MINC-drug is not an antibiotic.

In one embodiment, the infection is caused by methicillin-resistant Staphylococcus aureus, and the drug is IFN-α, IFN-γ, IFN-β, IL-13 topoisomerase inhibitor, FtsZ inhibitor, beta-lactamase inhibitor, ribosome inhibitor, dihydropteroate synthase inhibitor, dihydropteroate synthase inhibitor, anti-AR301, anti-MEDI4893, anti-514G3, or anti-ARN-100.

In one embodiment, the infection is caused by Pseudomonas, and the drug is IFN-α, OprF vaccine, anti-LPS, anti-alginate, anti-PcrV, anti-OPK, anti-DNABII, beta-lactamase inhibitor, LptD, LpxD, bacteriolytic agent, Iron mimic agent, biofilm matrice disruptor, T3SS inhibitor, anti-MEDI3902, or anti-AR101.

In one embodiment, the infection is caused by tuberculosis, and the drug is IFN-α, anti-TNF-α, IFN-γ, GM-CSF, TGF-β, anti-VEGF, IL-2, IL-4, IL-7, IL-10, IL-12, IL-15, IL-23, IL-24, IL-37, anti-IL4 prostaglandin E, Phosphodiesterase inhibitors, ATP synthase inhibitor, ribosome inhibitor, DprE1 inhibitor, Gyrase inhibitor, MmpL3 inhibitor, Leu tRNA synthase inhibitor, Murl inhibitor, InhA inhibitor, NDH-2 inhibitor, QcrB inhibitor, MenG inhibitor, FtsZ inhibitor, EthR inhibitor, or LepB inhibitor.

In one embodiment, the infection is caused by Bacillus anthracis, and the drug is IFN-α, Anti-PA63, Enoyl-ACP Reductase, Ribonucleotide Reductase, Nucleoside Hydrolase, Replicative DNA Helicases, Acetohydroxyacid Synthase, NAD Synthase, Nicotinic Acid Mononucleotide Adenylyltransferase, Lumazine Synthase, Cytoskeleton Protein FtsZ, Dihydropterate Synthase, or dihydrofolate reductase.

In one embodiment, the infection is caused by Tetani bacterium, and the drug is IFN-α, anti-tetanus immunoglobulin, tetanus toxoid, benzodiazepines, magnesium sulfate, intrathecal baclofen, dantrolene, ketamine, propofol, botulinum toxin, human anti-tetanus immunoglobulin, or tetanus toxoid vaccine.

In one embodiment, the infection is caused by Streptococcus pneumoniae, and the drug is IFN-α, IL-26, HMGB1, anti-IL-6, anti-TNFα, anti-IL-1β, anti-CXCL8, CD4, IL-4, CD8A, IL-10, anti-PTPRC, JAK inhibitors, anti-manL, anti-cps4L, anti-recU, anti-SP_0645, anti-ezrA, anti-prsA, anti-tarJ, anti-SP_1280, anti-SP_1617, anti-ptsG, anti-DltD, anti-hprK, anti-pepF, anti-coiA, anti-fib, anti-acpS, anti-manA, anti-mvaK2, anti-mtlD, anti-mtlF, or TPCA-1.

In one embodiment, the infection is caused by meningococcus, and the drug is IFN-α, anti-IL-1β, anti-TNFα, anti-IL-8, anti-MIP-2, anti-MIP-1α, anti-MMP, anti-TGF-β, CGRP, anti-PARP, TACE, EGFR, EGF, anti-ATM, ESR-1, anti-CASP8, NGF, anti-sdhA, anti-ribH, anti-ruvA, anti-ruvX, anti-ponA, anti-rr03, anti-fabH, anti-fabZ, anti-metE, anti-recJ, anti-rpsP, anti-plsY, anti-ftsK, anti-dnaE, anti-holB, anti-rsmI, anti-mtf, anti-dnaG, anti-rpoD, anti-pta, anti-rplU, anti-hup, anti-ptsI, anti-rsmG, anti-lgt, anti-greA, anti-secA1, anti-queF, anti-nusG, anti-ackA, anti-dapH, anti-ilvD, or anti-dnaC.

In one embodiment, the infection is caused by severe acute respiratory syndrome coronavirus 2, and the drug is IFN-α, IFN-β, IL-6, IL-12, IL-21, anti-IL-6, anti-IL-6R, Spike vaccine, RBD vaccine, ACE2 antagonist, anti-TMPRSS2, anti-CD147, anti-VEGFA, anti-GM-CSF, dexamethasone, chloroquine, Nsp12-RdRp inhibitor, hydroxychloroquine, anti-3CLpro, interferon-α, AAK-1 inhibitor, or JAK inhibitor.

In one embodiment, the infection is caused by enterovirus virus, and the drug is IFN-α, IFN-β, Capsid binder, 3Cpro inhibitor, 3Dpol inhibitor, 2CATPase inhibitor, 2APro inhibitor, anti-HSP90, anti-PI4KB, anti-OSBP, anti-RdRP, SP40, SP45, SP55, SP81, LVLQTM, VAD, or AAPV.

In one embodiment, the infection is caused by HIV, and the drug is IFN-α, anti-tetanus immunoglobulin, tetanus toxoid, benzodiazepines, magnesium sulfate, intrathecal baclofen, dantrolene, ketamine, propofol, botulinum toxin, human anti-tetanus immunoglobulin, or tetanus toxoid vaccine.

In one embodiment, the infection is caused by hepatitis B virus, and the drug is IFN-α, IFN-γ, IL-12, IL-6, IL-21, DNA polymerase reverse transcriptase activity inhibitor, preS1 peptide, CRISPR/Cas9, ZFNs, capsid assembly modulator, E-neg and E-pos RNAi, Lamivudine, adefovir, entecavir, Tenofovir, TLR agonist, STING agonist, or cyclophilin inhibitor.

In one embodiment, the infection is caused by MERS-CoV, and the drug is IFN-α, IFN-β, IFN-γ, anti-DPP4, Poly IC, anti-ACE2 antagonist, RdRp inhibitor, Chlorpromazine hydrochloride, Chloroquine, Peptide, endosome protease inhibitor, TMPRSS2 inhibitor, Furin protease inhibitor, anti-clathrin endocytosis, MERS-CoV S DNA, or RBD subunit vaccine.

In one embodiment, the infection is caused by influenza virus, and the drug is IFN-α, IFN-β, IFN-γ, IL-12, IL-6, IL-15, IL-21, neuraminidase inhibitor, cap dependent endonuclease inhibitor, M2 ion channel blockers, nucleoprotein inhibitor, anti-NS1-1, anti-CPSF30, anti-PABII, anti-eIF4G1, anti-PABP1, anti-p85, anti-PKR, anti-PACT, anti-NXF1, anti-p15, anti-Importin, anti-crk, anti-crkL, anti-RIG-1, anti-nucleolin, anti-TRIM25, anti-Gas8, anti-Akt, anti-p53, antiPARP10, anti-RIL, anti-Hsp90, anti-PDZ, anti-NOLC1, anti-RAP55, anti-IKK, anti-hPAF1C, or anti-hGBP1.

In one embodiment, the infection is caused by Dengue virus, and the drug is anti-TNF-α, anti-IL-6, anti-RANTES, Chloroquine, Prednisolone, NS5 nucleoside inhibitor, ER-associated α, Glucosidase inhibitor, Lovastatin, capsid inhibitor, envelope inhibitor, anti-NS4B, anti-NS2B/3, anti-NS1, or NS1 vaccine.

In one embodiment, the infection is caused by respiratory syncytial virus, and the drug is IL-15, anti-TNF-α, Benzimidazole derivatives, disulfonated stilbene, imidazoiso-indolone derivative, triphenolic compound, anti-envelope glycoproteins, sulfated sialyl lipid, anti-NS1, anti-F glycoprotein, siRNA inhibit P protein, NS1 protein, or N protein genes.

In one embodiment, the infection is caused by SARS-CoV, and the drug is IFN-α, IFN-β, IL-21, anti-IL-6, anti-IL-6R, anti-VEGFA, anti-GM-CSF, Spike vaccine, RBD vaccine, ACE2 antagonist, anti-TMPRSS2, anti-CD147, dexamethasone, chloroquine, Nsp12-RdRp inhibitor, hydroxychloroquine, anti-3CLpro, Interferon-a, AAK-1inhibitor, JAK inhibitor, anti-ATM, anti-SIRT1, anti-GSK3B, or anti-Torin-2.

In one embodiment, the infection is caused by Escherichia coli, and the drug is IFN-α, anti-IL-1β, anti-TNFα, anti-IL-6, anti-bamD, anti-cydX, anti-dnaT, anti-fabA, anti-ftsB, anti-ftsL, anti-ftsQ, anti-hemD, anti-higA, anti-hipB, anti-holD, anti-iraM, anti-lolA, anti-lolB, anti-lptA, anti-lptD, anti-lptE, anti-mreD, anti-mukB, anti-mukE, anti-mukF, anti-pheM, anti-priB, anti-safA, anti-secE, anti-trpL, anti-tusE, anti-wzyE, anti-ycaR, anti-yciS, anti-ydfO, anti-ydhL, anti-ygfZ, anti-yqeL, anti-yrfF or anti-zipA.

In one embodiment, the infection is caused by Legionella, and the drug is IFN-α, anti-IL-1β, anti-TNFα, anti-IL-8, anti-MIP-2, anti-MIP-1α, anti-MMP, anti-TGF-β, Anti-Hsp60, Anti-MOMP, Anti-Mip, Anti-Les, Anti-Lsp, Anti-CpxTRA, Anti-PilEL, Anti-Lvh, Anti-IcM, Anti-UDP, Anti-FtsL, Anti-MrdA, Anti-RpoH, Anti-Multidrug resistance protein, Anti-Amino acid permease, Anti-TolB, Anti-Kup1, Anti-MviN, Anti-Tig, Anti-MurE, Anti-SCD, Anti-TTF, Anti-UMF1, Anti-HMP, Anti-HslU, Anti-htrB, Anti-Kdo, Anti-OGT or Anti-RpoH.

In one embodiment, the infection is caused by Neisseria gonorrhoea, and the drug is IFN-α, anti-IL-1β, anti-TNFα, anti-IL-8, anti-MIP-2, anti-MIP-1α, anti-MMP, anti-TGF-β, CDP-4-dehydro-6-deoxyglucose reductase, Anti-LpxC, Anti-SSADH, Anti-RNR, Anti-DacC, Anti-PBP, Anti-PIB, Anti-dsbA, Anti-NarX, Anti-Zur, Anti-PTS, Anti-Hpr, Anti-PPP, Anti-YgfZ, Anti-HP, Anti-RimM, Anti-BspRI, Anti-rluF, Anti-FAD, Anti-EF-P, Anti-dnaN, Anti-tilS or Anti-RluD.

In one embodiment, the infection is caused by Neisseria meningitidis, and the drug is IFN-α, anti-IL-1β, anti-TNFα, anti-IL-8, anti-MIP-2, anti-MIP-1α, anti-MMP, anti-TGF-β, Anti-TerC, Anti-MscS, Anti-OPT, Anti-MFS, Anti-MscS, Anti-NaCT, Anti-ABC, Anti-TauE/SafE, Anti-CBS, Anti-NRAMP, Anti-OATP, Anti-YggX, Anti-SstT, Anti-PMT, Anti-YjgP, Anti-yhhQ, Anti-TerC, Anti-MAPEG, Anti-MSG, Anti-PilW, Anti-PilX, Anti-RlpA, Anti-Rrf2, Anti-IclR or Anti-Rim.

In one embodiment, the infection is caused by Salmonella, and the drug is IFN-α, anti-IL-1β, anti-TNFα, anti-IL-6, anti-bamD, anti-cydX, anti-dnaT, anti-fabA, anti-ftsB, anti-ftsL, anti-ftsQ, anti-hemD, anti-higA, anti-hipB, anti-holD, anti-iraM, anti-lolA, anti-lolB, anti-lptA, anti-lptD, anti-lptE, anti-mreD, anti-mukB, anti-mukE, anti-mukF, anti-pheM, anti-priB, anti-safA, anti-secE, anti-trpL, anti-tusE, anti-wzyE, anti-ycaR, anti-yciS, anti-ydfO, anti-ydhL, anti-ygfZ, anti-yqeL, anti-yrfF or anti-zipA.

In one embodiment, the infection is caused by hepatitis C virus, and the drug is IFN-α, IFN-β, IL-21, anti-IL-6, anti-IL-6R, Anti-EGFR, Anti-NS2, Anti-NS3, Anti-NS4A, Anti-NS5A, Anti-NS5B, Anti-Helicase, Anti-TLR-9, Anti-HCV E2, Anti-E1, Anti-E2, Anti-p7, Anti-CD81, Anti-SRB1, Anti-CLDN1, Anti-EphA2, Anti-TfR1, Anti-NPC1L1, Cyclosporin A, Anti-Alpha-glucosidase, Anti-DGAT-1, Anti-VLDL, Anti-CD81, Anti-CLDN1 or Anti-SR-B1.

In one embodiment, the infection is caused by monkeypox virus, and the drug is IFN-α, IFN-β, IL-21, anti-IL-6, anti-IL-6R, Rosmarinic acid, Myricitrin, Quercitrin, Ofloxacin, Anti-VP37, Anti-F13L, Anti-E9L, Anti-A24R, Anti-A48R, Anti-H5R, Anti-BIR, Anti-F10L, Anti-E8L, Anti-A6R, Anti-SPGF, Anti-B8R, Anti-A50R, Anti-I7L, Anti-D13L, Anti-Top1 or Anti-TMPK.

In one embodiment, the infection is caused by human papillomavirus, and the drug is IFN-α, IFN-β, IL-21, anti-IL-6, anti-IL-6R, Anti-EGFR, Anti-c-Met, Anti-IGF-1R, Anti-PI3K, Anti-Akt, Anti-mTOR, Anti-Ras, Anti-Raf, Anti-MAPK, Anti-VEGF, Anti-VEGFR, Anti-HIF-1alpha, anti-PD-L1, anti-CD38, anti-PD1, anti-RNR, anti-FGFR, anti-PDGFR, anti-Kit or anti-Ret.

Dosing of the MINC-drug is based on the known dosage of the drug for treating a particular disease and the subject condition. The dosage can be a Food and Drug Administration (FDA) approved dosage or a dosage used in clinical trial.

In MINC-drug, in general, the dosage of PEG-EGCG combined with OEGCG is between 10 μg/kg to 100 mg/kg.

The concentration for the encapsulated drug agents can be as low as 0.01 μg/kg (e.g., for cytokine drugs, IFN-α) and as high as 500 mg/kg (e.g., for antibody drugs, atoltivimab).

For example, for treating SARS-CoV-2, HIV, HPV or other viral infection in an adult human, IFN-α is administered 0.01-500 μg/kg IV (or 0.01-500 mIU) once to three times every week. The same effective dose of MINC-IFN-α can be used for treating other viral infection.

For example, for treating Mycobacterium tuberculosis, Clostridium tetani, Salmonella Typhi, Corynebacterium diphtheria, Treponema pallidum, Mycobacterium lepromatosis, Methicillin-resistant Staphylococcus aureus or other bacterial infection in an adult human, IFN-α is administered 0.01-500 μg/kg IV (or 0.01-500 mIU) once to three times every week. The same effective dose of MINC-IFN-α can be used for treating other bacterial infection.

For example, for treating SARS-CoV-2, HIV, HPV or other viral infection in an adult human, IFN-β is administered 0.01-500 μg/kg IV (or 0.01-500 mIU) once to three times every week. The same effective dose of MINC-IFN-β can be used for treating other viral infection.

For example, for treating Mycobacterium tuberculosis, Clostridium tetani, Salmonella Typhi, Corynebacterium diphtheria, Treponema pallidum, Mycobacterium lepromatosis, Methicillin-resistant Staphylococcus aureus or other bacterial infection in an adult human, IFN-β is administered 0.01-500 μg/kg IV (or 0.01-500 mIU) once to three times every week. The same effective dose of MINC-IFN-β can be used for treating other bacterial infection.

In general, when a MINC-drug has the drug being a cytokine such as IL-1, IL-2, IL-3, IL-4, IL-5, IL-6, IL-7, IL-8, IL-10, IL-12, IL-13, IL-15, IL-17, IL-18, IFN-α, IFN-β, TNF-α, G-CSF, GM-CSF or M-CSF for treating viral and bacterial infection, the cytokine can be administered at a range of 0.01-500 μg/kg IV (or 0.01-500 mIU) once to three times every week.

For example, for treating SARS-CoV-2 in an adult human, Bamlanivimab is administered 10-2000 mg IV once. The effective dose of MINC-Bamlanivimab in the same dose range can be used for treating SARS-CoV-2.

For example, for treating HIV in an adult human, Ibalizumab is administered 10-6000 mg IV every two weeks. The effective dose of MINC-Ibalizumab in the same dose range can be used for treating HIV.

For example, when a MINC-drug having the drug being an antibody including but not limited to f-CoV-2-RBD, Anti-SARS-CoV-2-SP, Anti-SARS-CoV-2-SD1/SD2, Anti-SARS-COV-2-S2, Bamlanivimab, Etesevimab, Casirivimab, Imdevimab, Cilgavimab, Tixagevimab, Sotrovimab, Regdanvimab, Anti-HIV-gp120, VRC01, 10-1074, Anti-HIV-Env, 3BNC117, Anti-HIV-gp41, 4E10, 2F5, 2G12, Anti-CCR5, PRO 140, Anti-CD4, Ibalizumab, Leronlimab, Anti-HPV-E6, Anti-HPV-E7, Anti-CMV-gB, CSJ148, TCN-202, Anti-CMV-gH, LPJ539, Anti-CMV-gH/gH, MCMV5322A, Sevirumab, MCMV3068A, Anti-influenza A hemagglutinins, MHAA4549A, VIS410, CR6261, CR8020, TCN-032, Anti-RSV-glycoprotein F, Palivizumab, REGN2222, Motavizumab, MEDI8897, ALX-0171, Nirsevimab, ZMapp, atoltivimab, maftivimab, odesivimab, CR57, CR4098 or CL184, RAB-1 for treating viral infection, the agent can be administered at a range of 0.1-500 mg/kg IV once daily to once every three weeks.

For example, when a MINC-drug having the drug being an antibody including but not limited to AR301, MEDI4893, 514G3, ARN-100, MEDI3902, AR101, PolyCAb, Cd-ISTAb, AR401-mAb, VXD-003, ASN-4 or ASN-5 for treating bacterial infection, the drug can be administered at a range of 0.1-500 mg/kg IV once daily to once every three weeks.

For example, for treating SARS-CoV-2 in an adult human, Remdesivir is administered 10-1000 mg IV once daily to once every other day. The effective dose of MINC-Remdesivir in the same dose range can be used for treating SARS-CoV-2 or other viral infection.

For example, for treating SARS-CoV-2, HBV, HCV, influenza virus or other viral infection in an adult human, Ribavirin is administered 50-10000 mg IV once daily to once every three days. The effective dose of MINC-Ribavirin in the same dose range can be used for treating SARS-CoV-2 or SARS-CoV-2, HBV, HCV, Influenza virus or other viral.

For example, when a MINC-drug has the drug being a nucleoside or nucleotide analogue including but not limited to Favipiravir, Lopinavir, Ritonavir, Acyclovir, Famciclovir, Valacyclovir, Abacavir, Didanosine, Emtricitabine, Stavudine, Tenofovir, Zidovudine, Adefovir, Emtricitabine, Entecavir, Lamivudine, Telbivudine, Cidofovir, Ganciclovir, Valganciclovir, Famciclovir, Valacyclovir, 5-azacytidine, and 5-fluorouracil, Zanamivir, Oseltamivir, Peramivir or Baloxavir for treating viral infection, the drug can be administered at a range of 0.5 to 25000 mg IV once to three times every week.

The present invention is useful in treating human and non-human animals. For example, the present invention is useful in treating a mammal subject, such as humans, horses, pigs, cats, and dogs.

The following examples further illustrate the present invention. These examples are intended merely to be illustrative of the present invention and are not to be construed as being limiting.

### EXAMPLES

### Active Ingredients for All Examples

### OEGCG

OEGCG is oligomerized EGCG. OEGCG was prepared according to WO2006/124000.

### PEG-EGCG

PEG-EGCG is PEG conjugated with one or two EGCG. PEG-EGCG was prepared according to WO2006/124000, WO2009/054813, or WO2015/171079.

### MINC-Drugs

MINC-drugs were made by the same way as MINC-doxorubicin according to WO2011/112156 or WO2015/171079. Alternatively, MINC-drugs can be prepared by encapsulated an agent within the micelle formed by PEG-EGCG and OEGCG, according to the method in WO2006/124000 or WO2009/054813.

### Example 1: OEGCG has Anti-Bacterial Activity Against MRSA

### Materials

OEGCG is oligomerized EGCG. OEGCG was prepared according to WO2006/124000.

### Method

To understand the efficacy of MINC platform in anti-bacterial therapy, OEGCG was used in treating methicillin-resistant Staphylococcus aureus (MRSA). OEGCG is prepared at 100 μg/mL. The concentration of bacterial suspension is around 1.0×10⁸˜1.0×10⁹ CFU/mL. After adding 0.1 mL of MRSA into 10 mL tested OEGCG and control sterile saline, the incubating procedure was followed “ASTM E2315-16, Standard Guide for Assessment of Antimicrobial Activity Using a Time-Kill Procedure” Each group was inoculated on medium and the growth was observed and colony counts are recorded. The data are presented as reduction rate=100×(1−10^{LR}); LR=Log (Control)−Log (OEGCG).

### Result

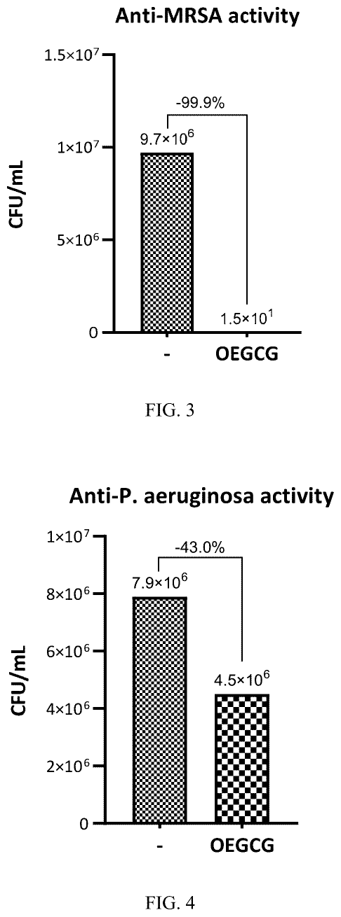

FIG. 3 shows that OEGCG reduced the colony formation of methicillin resistant Staphylococcus aureus (MRSA), showing its efficacy in suppressing MRSA growth. Staphylococcus belongs to gram positive bacteria and the results demonstrate the efficacy in treating this group of bacteria.

### Example 2: OEGCG has Anti-Bacterial Activity Against Pseudomonas Aeruginosa

### Materials

### Method

To understand the efficacy of MINC platform in anti-bacterial therapy, OEGCG was used in treating Pseudomonas aeruginosa. OEGCG was prepared at 100 μg/mL. The concentration of bacterial suspension was around 1.0×10⁸˜1.0×10⁹ CFU/mL. After adding 0.1 mL of Pseudomonas aeruginosa into 10 mL tested OEGCG and control sterile saline, the incubating procedures were followed according to “ASTM E2315-16, Standard Guide for Assessment of Antimicrobial Activity Using a Time-Kill Procedure”. Each group was inoculated on medium and the growth was observed and colony counts were recorded. The data is presented as reduction rate=100×(1−10LR); LR=Log (Control)−Log (OEGCG).

### Result

FIG. 4 shows that OEGCG reduced the colony formation of Pseudomonas aeruginosa, suggesting its efficacy in suppressing Pseudomonas aeruginosa growth. Pseudomonas belongs to gram negative bacteria and the results demonstrate the efficacy in treating this group of bacteria.

### Example 3: OEGCG and PEG-EGCG Inhibit SARS-CoV-2 Infection

### Materials

Human coronavirus (nCOV-Luc-D614G), as a surrogate for SARS-CoV-2, was obtained from Academia Sinica siRNA core.

HEK293T-hACE2 cells were generated by transduction of VSV-G pseudotyped lentivirus carrying human ACE2 gene in HEK-293T/17 (ATCC® CRL-11268TM)

### Method

HEK293T-hACE2 cells were seeded in 1×10⁴ in 96 well plates using Beckman Biomek i5 liquid handling system. In Day1: Cells were seeded and incubated at 37° C. in a CO₂ incubator for 16˜18 hrs. The next day, 100 μL of serial diluted OEGCG or PEG-EGCG were added to 4,000 TU SARS-CoV-2 pseudovirus in a 100 μL culture medium. Each OEGCG and PEG-EGCG were incubated with virus for 1 hour at 37° C. After the incubation, 50 μL of the mixture was added to pre-seeded 50 μL of HEK293T-hACE2 cells and incubated at 37° C. for 24 hours. After 24 hours, 80 μL medium was removed and 50 μL of culture medium (DMEM with 10% FBS) was added to the 96 well plates. At 72 hours post-infection, 50 μL Bright-Glo-Luciferase reagent was added to each well and mixed evenly by using Beckman liquid handling system (Beckman program: white plate ace2-Day5—Luciferase-3 or 6 plate-deep well-SPL). The related light unit (RLU) was detected by a microplate reader, Tecan Infinite F500 (Program: Luminescence Nunc White96_100 ms)

### Results

The anti-SARS-CoV-2 potency of OEGCG was shown by ID50 and ID90 values of 13.5 nM and 24.6 nM, respectively (FIG. 5A). The anti-SARS-CoV-2 activity of PEG-EGCG was measured at a concentration of 5 μM and 20 μM, which exhibits 18.4% and 28.3% inhibition (FIG. 5B). The results demonstrate both OEGCG and PEG-EGCG have anti-viral activity against SARS-CoV-2. [No, I do not agree with conclusion]

### Example 4: OEGCG Inhibits Enterovirus Infection

### Materials

Rhabdomyosarcoma (RD) cells were obtained from ATCC CCL-136TM

### Method

To evaluate the anti-viral activity of OEGCG against EV71. RD cells were seeded at 2×10⁴ cells per well in 96 well plate and incubated at 37° C. for 16˜18 hours. For virus infection, different concentrations of OEGCG were co-incubated with 0.1 multiplicity of infection (MOI) of EV-D68 virus in the 96 well plate with RD cells at 33° C. or 37° C. for 1 hour. 48 hours after infection, the cell viability was examined using MTT (3-(4,5-dimethylthiazol-2-yl)-2,5-diphenyl tetrazolium bromide) assay (Sigma-Aldrich, USA). The absorbance (O.D) was read at 570 nm using microplate reader. Antiviral activity (%) was calculated as [(O.D. value of OEGCG treated sample)−(O.D. value of virus only)]/(O.D. value of virus only)×100%

### Result

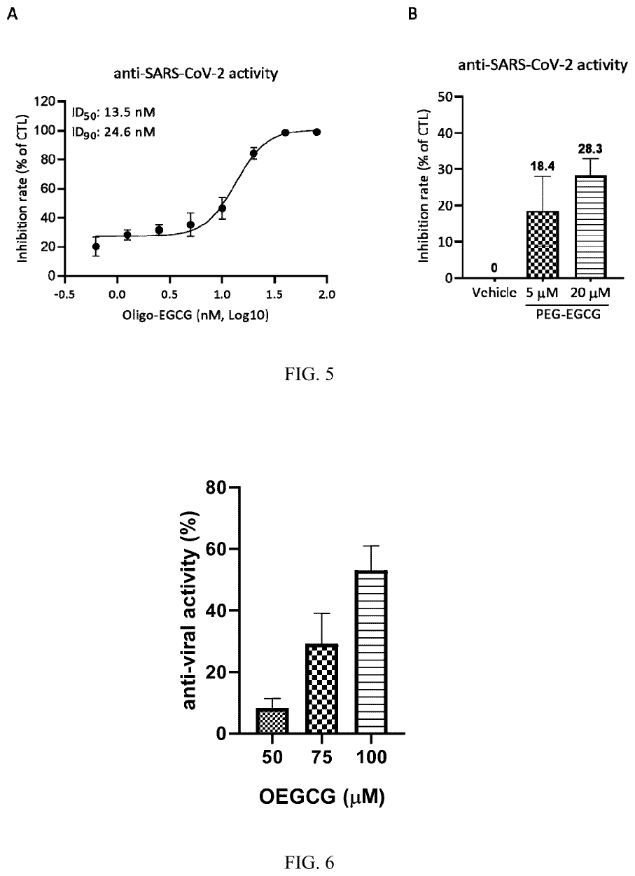

The results demonstrate that OEGCG treatment inhibited EV D68 infection (FIG. 6). The antiviral activity of OEGCG showed a dose-dependent increase, with corresponding percentages of 8.3%, 29.2%, and 53.1% at 50 μM, 75 μM, and 100 μM, respectively.

### Examples 5-6 Show Different Flavonoid Oligomers and Polymer-Flavonoid Conjugates Formed MINC-Drugs

### Example 5: Method for Preparing MINC-Anti-HER2 Using Different Flavonoids in Flavonoid Oligomers and Polymer-Flavonoid Conjugates

### Materials

Anti-HER2 is trastuzumab obtained from Eirgenix.

### Method

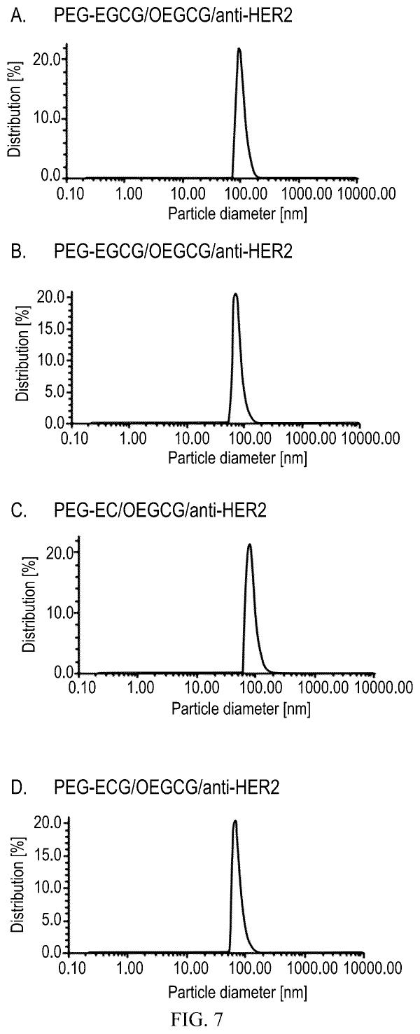

MINC anti-HER2 nanoparticles were prepared according to WO2009/054813. In brief, anti-HER2 was incubated in PBS. Subsequently, different flavonoid oligomer including OEGCG or OECG was added to anti-HER2, followed by adding different polymer-flavonoid including PEG-EGCG, PEG-ECG or PEG-EC. After incubating the mixture at room temperature, 10K MWCO centrifugal filter was used to remove the unreacted oligomer flavonoid and polymer-flavonoid. DLS (Anton Paar Litesizer 500) was used to measure the nanoparticle size and the results are shown in FIG. 7.

### Results

FIG. 7 shows that different polymer-flavonoid conjugate and different flavonoid oligomers all successfully generated MINC-anti-HER2 micelles with particle size around 100 nm. The results demonstrate that homogenous nanoparticles (micelles) were formed with an expected size around 100 nm, which is different from unencapsulated anti-HER (about 5-10 nm).

In this example, different flavonoid oligomers used were OEGCG (FIGS. 7A, 7C and 7D) and OECG (FIG. 7B); and different polymer-flavonoids used were PEG-EGCG (FIGS. 7A and 7B), PEG-EC (FIG. 7C) and PEG-ECG (FIG. 7D).

These data support that MINC nanoparticle can be formed by different flavonoid oligomers and different polymer-flavonoid conjugates.

### Example 6: Method for Preparing MINC-BSA Using Different Polymers and Different Polymer-Flavonoid Conjugates

### Materials

BSA was purchased from Sigma-Aldrich.

### Method

MINC (Multi-target Immune Nanocarrier Combination)-BSA nanoparticles were prepared according to WO2009/054813. In brief, BSA was incubated in PBS. Subsequently, OEGCG or OEGCG was added to BSA, followed by adding different polymer-flavonoid including PEG-EGCG, HA-EGCG and Dextran-EGCG. After incubating the mixture at room temperature, 10K MWCO centrifugal filter was used to remove the unreacted OEGCG and polymer-flavonoid. DLS (Anton Paar Litesizer 500) was used to measure the nanoparticle size.

### Results

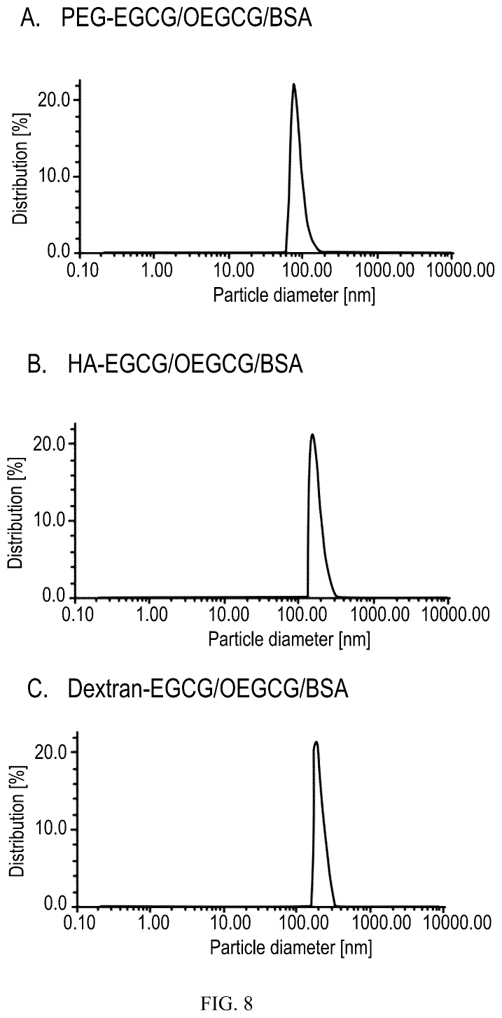

FIG. 8 demonstrates that different polymers in polymer-flavonoid conjugates can be used to successfully generate MINC-BSA. The results demonstrate that homogenous nanoparticles (micelles) were successfully formed, and there was no small peak (about 5-10 nm) of unencapsulated BSA observed. These different polymers were PEG (FIG. 8A), HA (FIG. 8B) and Dextran (FIG. 8C). Altogether, these data support that MINC nanoparticle can be formed by different polymer-flavonoids conjugates.

Examples 7-13 show successful formation of MINC-IFN-α, MINC-IFN-γ, MINC-IL-12, MINC-IL-2, MINC-IL-6, MINC-IL-15, and MINC-IL-21.

OEGCG and PEG-EGCG used in Examples 8-13 were prepared by the same protocols as described in Example 7.

### Example 7. MINC-IFNα Formulation

### Materials

IFN-α was purchased from PharmaEssentia.

### Method

MINC-IFN-α nanoparticles were prepared according to Example 5. DLS (Malvern Zetasizer Nano ZS) was used to measure the size of MINC-IFN-α nanoparticles

### Results

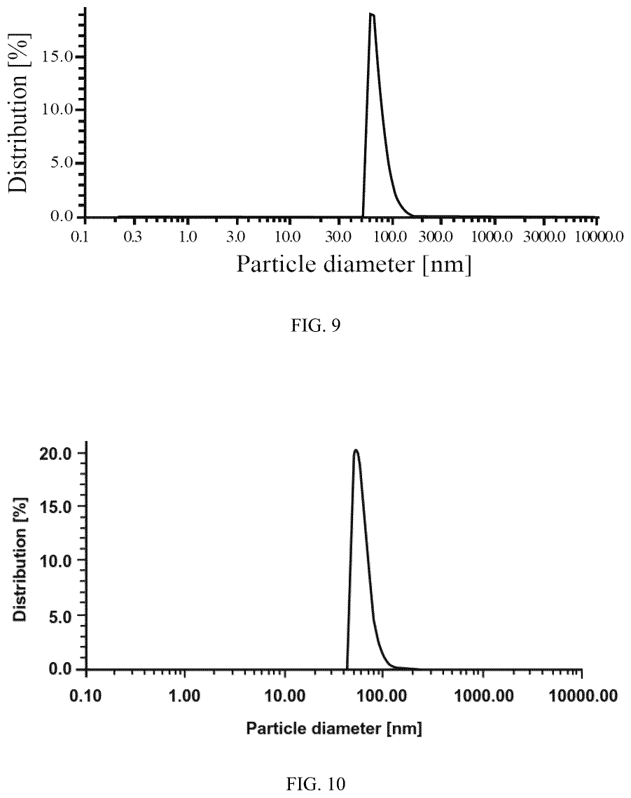

FIG. 9 shows a successful formulation of MINC-IFN-α.

### Example 8. MINC-IFN-γ Formulation

### Materials

IFN-γ was purchased from BioLegend.

### Method

MINC-IFN-γ nanoparticles were prepared according to Example 5. DLS (Anton Paar Litesizer 500) was used to measure the size of MINC-anti-IFN-γ nanoparticles

### Results

FIG. 10 shows a successful formulation of MINC-IFN-γ.

### Example 9. MINC-IL-12 Formulation

### Materials

IL-12 was purchased from BioLegend.

### Method

MINC-IL-12 nanoparticles were prepared according to Example 5. DLS (Anton Paar Litesizer 500) was used to measure the size of MINC-anti-IL-12 nanoparticles

### Results

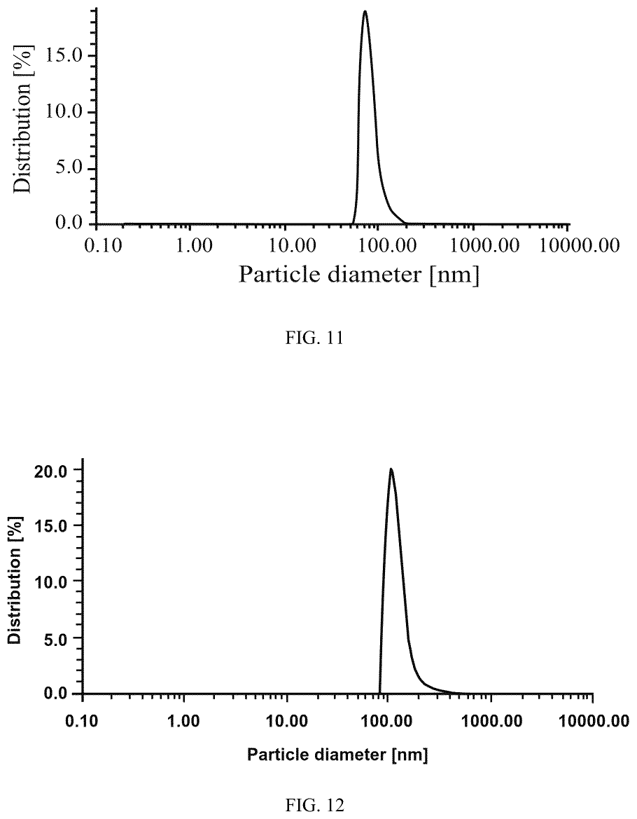

FIG. 11 shows a successful formulation of MINC-IL-12.

### Example 10. MINC-IL-2 Formulation

### Materials

IL-2 was purchased from BioLegend.

### Method

MINC-IL-2 nanoparticles were prepared according to Example 5. DLS (Anton Paar Litesizer 500) was used to measure the size of MINC-anti-IL-2 nanoparticles

### Results

FIG. 12 shows a successful formulation of MINC-IL-2.

### Example 11. MINC-IL-6 Formulation

### Materials

IL-6 was purchased from BioLegend.

### Method

MINC-IL-6 nanoparticles were prepared according to Example 5. DLS (Anton Paar Litesizer 500) was used to measure the size of MINC-anti-IL-6 nanoparticles.

### Results

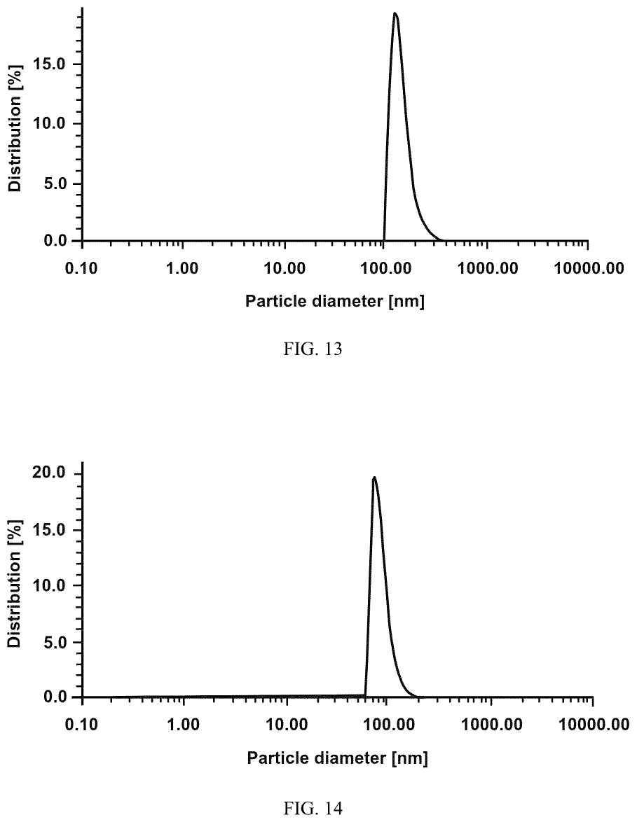

FIG. 13 shows a successful formulation of MINC-IL-6.

### Example 12. MINC-IL-15 Formulation

### Materials

IL-15 was purchased from BioLegend.

### Method

MINC-IL-15 nanoparticles were prepared according to Example 5. DLS (Anton Paar Litesizer 500) was used to measure the size of MINC-anti-IL-15 nanoparticles

### Results

FIG. 14 shows a successful formulation of MINC-IL-15.

### Example 13. MINC-IL-21 Formulation

### Materials

IL-21 was purchased from BioLegend.

### Method

MINC-IL-21 nanoparticles were prepared according to Example 5. DLS (Anton Paar Litesizer 500) was used to measure the size of MINC-anti-IL-21 nanoparticles

### Results

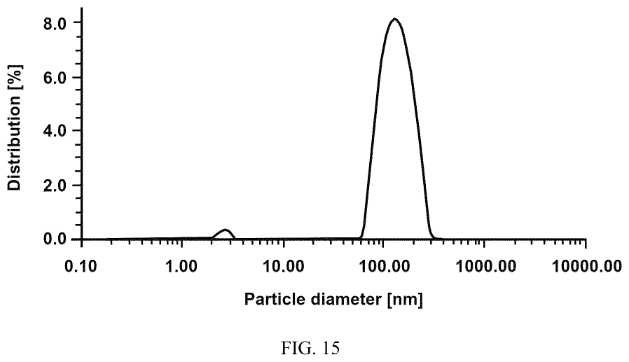

FIG. 15 shows a successful formulation of MINC-IL-21.

### Example 14. MINC-IFN-α Inhibits SARS-CoV-2 Replication (Prophetic Example)

### Material

Vero E6 cells (American Type Culture Collection, Manassas, VA) is used.

SARS-CoV-2 (Wuhan strain or BA.1) is used.

### Method

The Cytopathic Endpoint Assay is used to evaluate anti-viral activity of MINC-IFN-α. Briefly, 100 μL of serial 10-fold dilutions of MINC-IFNα or IFNα are incubated with 100 μL of Vero E6 cells, giving a final cell count of 20,000 cells per well in a 96-well plate. The cells are incubated at 37° C. in 5% CO2 overnight. On the second day, ten μL of virus at a concentration of 10,000 PFU/well is then added to each of the test wells. The plates are incubated at 37° C. in 5% CO₂ for 3 days to observe daily for cytopathic effect (CPE). The end point is the drug dilution that inhibited 100% of the CPE in quadruplicate wells.

### Results

MINC-IFN-α prevents Vero cell death (CPE) from SARS-CoV-2 infection in this study. We expect MINC-IFN-α has lower IC50 (50% inhibition concentration) than IFN-α alone at equivalent IFN-α concentration.

### Example 15: MINC-IFN-α Inhibits MRSA Proliferation (Prophetic Example)

### Material

S. aureus strain LAC (a pulsed-field type USA300 strain) is used.

### Method

For MRSA killing assays, 4×10⁵ neutrophils are incubated in RPMI medium with PBS, MINC-IFN-α or IFN-α (100 ng/ml), followed by the addition of 50 μl of MRSA (at a 1:1800 dilution of an optical density at 600 nm (OD₆₀₀)=0.25) pre-incubated in 10% autologous serum. At the desired time points, saponin (22 μl of a 1% solution, 0.1% final concentration) is added to each well or tube, the contents are mixed, and the plates or tubes are incubated on ice for 15 min. MRSA are subsequently passed through a 25-gauge blunt-end needle (to disperse cell clumps) and plated on LB agar. Surviving bacteria are enumerated the following day. Percent survival is calculated by comparing the numbers of surviving bacteria to those at t=0 with the equation: (CFU+at t/CFU−at t₀)×100.

### Results

MINC-IFN-α promotes neutrophils phagocytosis activity to clear MRSA. MINC-IFN-α treatment is expected to decrease MRSA colony formation numbers compared to vehicle control (saline). We expect MINC-IFN-α has better reduction in colony number than IFN-α alone at equivalent IFN-α concentration.

### LIST OF ABBREVIATIONS

2APro 2A protease
2CATPase 2C-like ATPase
3CLpro 3-chymotrypsin-like protease
3Cpro 3C protease
3Dpol Poliovirus RNA-dependent RNA Polymerase
AAK-1 AP2-associated protein kinase 1
ABC ATP-binding cassette
Akt Protein kinase B (PKB)
ALB Albumin
ATM Ataxia-telangiectasia mutated
CASP8 Caspase 8
CBS Cystathionine beta-synthase
CD147 Cluster of differentiation 147
CD38 Cluster of differentiation 38
CD4 Cluster of differentiation 4
CD81 Cluster of differentiation 81
CGRP Calcitonin gene-related peptide
CLDN1 Claudin-1
c-Met Hepatocyte growth factor receptor
CPSF30 Cleavage and polyadenylation specificity factor subunit 30
CpxTRA Compromised outer membrane protein transporter
crk Proto-oncogene C-crk
crkL Proto-oncogene C-crk-like
CXCL8 C-X-C motif chemokine ligand 8
DacC D-alanyl-D-alanine carboxypeptidase
DGAT-1 Diacylglycerol O-acyltransferase 1
DNABII DNA-binding proteins from starved cells
DprE1 Decaprenylphosphoryl-β-D-ribose 2-epimerase 1
EGF Epidermal growth factor
EGFR Epidermal growth factor receptor
eIF4G1 Eukaryotic translation initiation factor 4 gamma 1
EphA2 Ephrin type-A receptor 2
ESR-1 Estrogen receptor 1
EthR Ethionamide resistance protein
F13L Vaccinia virus protein F13L
FAD Flavin adenine dinucleotide
FGFR Fibroblast growth factor receptor
FtsL Cell division protein FtsL
FtsZ Cell division protein FtsZ
Gas8 Growth arrest-specific 8
GSK3B Glycogen synthase kinase 3 beta
hGBP1 Human guanylate-binding protein 1
HIF-1alpha Hypoxia-inducible factor 1 alpha
HMGB1 High mobility group box 1 protein
HMP High-molecular-weight penicillin-binding protein
HP Haptoglobin
hPAF1C human PAF1 complex
Hpr Histidine-containing phosphocarrier protein
HslU ATP-dependent protease HslU
HSP60 Heat shock protein 60
HSP90 Heat shock protein 90
IcM Immunoglobulin mu chain
IFN-alpha Interferon alpha
IGF-1R Insulin-like growth factor 1 receptor
IKK I-kappa-B kinase
IL-1 Interleukin 1
Importin Karyopherin alpha
InhA Enoyl-[acyl-carrier protein] reductase [NADH]
JAK Janus kinase
Kit tyrosine-protein kinase KIT
Kup1 Copper transport protein Kup
LepB Leptospiral outer membrane protein B
Les Lactate efflux system protein
Leu Leucine
LptD Lipopolysaccharide transport protein D
LpxD UDP-3-O-[3-hydroxymyristoyl] glucosamine N-acyltransferase
Lsp Signal peptidase II
Lvh Leptospiral virulence factor Lvh
LVLQTM Uncharacterized protein LVLQTM
MAPEG Microsomal glutathione S-transferase
MAPK Mitogen-activated protein kinase
MEDI3902 Monoclonal antibody MEDI3902
MenG 1,4-dihydroxy-2-naphthoate octaprenyltransferase
MFS Major facilitator superfamily
Mip Macrophage infectivity potentiator
MIP-2 Macrophage inflammatory protein-2
MMP Matrix metalloproteinase
MmpL3 Mycobacterial membrane protein Large 3
MOMP Major outer membrane protein
MrdA Multiple resistance and pH adaptation protein A
MSG Major surface glycoprotein
mTOR Mechanistic target of rapamycin
MurE UDP-N-acetylmuramoyl-L-alanine-D-glutamate ligase
Murl Muramyl pentapeptide synthetase
MviN Lipid II flippase
NaCT Sodium-coupled citrate transporter
NarX Nitrate/nitrite response regulator NarX
NDH-2 NADH-quinone oxidoreductase subunit 2
NGF Nerve growth factor
NOLC1 Nucleolar and coiled-body phosphoprotein 1
NPC1L1 Niemann-Pick C1-like 1 protein
NRAMP Natural resistance-associated macrophage protein
NS1 Non-structural protein 1
Nsp12 RNA-dependent RNA polymerase
NXF1 Nuclear RNA export factor 1
OATP Organic anion transporting polypeptide
OGT O-linked N-acetylglucosamine transferase
OPK O-polysaccharide kinase
OprF Pseudomonas outer membrane porin F
OPT Oligopeptide transporter
OSBP Oxysterol-binding protein
p15 Cyclin-dependent kinase inhibitor 2B
p53 Tumor protein p53
p85 Phosphatidylinositol 3-kinase regulatory subunit alpha
PA63 Protective antigen
PABII Poly(A)-binding protein 2
PABP1 Poly(A)-binding protein 1
PACT Protein activator of interferon-induced protein kinase EIF2AK2
PARP Poly(ADP-ribose) polymerase
PARP10 Poly(ADP-ribose) polymerase family member 10
PBP Penicillin-binding protein
PcrV Pseudomonas aeruginosa type III secretion needle tip protein
PD1 Programmed cell death protein 1
PDGFR Platelet-derived growth factor receptor
PD-L1 Programmed death-ligand 1
PDZ PSD-95/Discs large/ZO-1 homology domain
PI3K Phosphoinositide 3-kinase
PI4KB Phosphatidylinositol 4-kinase beta
PIB Peptidoglycan-associated lipoprotein
PilEL Pseudomonas aeruginosa type IV pilin-associated protein
PilW Pseudomonas aeruginosa type IV pilus assembly protein
PKR Protein kinase R
PMT Peptide N-glycanase and O-glycosidase
PPP Protein phosphatase
PTPRC Protein tyrosine phosphatase receptor type C
PTS Phosphotransferase system
QcrB Quinol oxidase subunit B
Raf Rapidly accelerated fibrosarcoma kinase
RAP55 RNA-binding protein 55
Ras Rat sarcoma protein
RdRp RNA-dependent RNA polymerase
RdRP RNA-dependent RNA polymerase
Ret Rearranged during transfection protein
RIG-1 Retinoic acid-inducible gene I
RIL Receptor-interacting protein-like
RNR Ribonucleotide reductase
RpoH RNA polymerase sigma-32 subunit
SCD Stearoyl-CoA desaturase
SP40 Simian virus 40
SP45 Simian parvovirus 45
SP55 Simian parvovirus 55
SP81 Simian parvovirus 81
SRB1 Scavenger receptor class B type 1
SR-B1 Scavenger receptor class B type 1
SSADH Succinic semialdehyde dehydrogenase
STING Stimulator of interferon genes protein
TACE TNF-alpha converting enzyme
TfR1 Transferrin receptor 1
TGF-β Transforming growth factor beta
Tig Trigger factor
TLR Toll-like receptor
TLR-9 Toll-like receptor 9
TMPK Thymidine monophosphate kinase
TMPRSS2 Transmembrane Protease, Serine 2
TNFα Tumor necrosis factor alpha
TolB Outer membrane protein TolB
TPCA-1 2-[(aminocarbonyl)amino]-5-(4-fluorophenyl)-3-thiophenecarboxamide
TRIM25 Tripartite motif-containing protein 25
TTF TATA-binding protein-associated factor
UDP Uridine diphosphate
UMF1 Ubiquitin-fold modifier 1
VAD Vascular adhesion domain
VLDL Very low-density lipoprotein
VP37 Viral protein 37

It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed is:

1. A method of treating a bacterial or viral infection, comprising the step of administering to a subject in need thereof an effective amount of micelles having an outer shell comprising one or more polymer-flavonoid conjugates, optionally an inner shell comprising one or more flavonoid oligomer, and a drug encapsulated within the shells;
wherein the polymer is a hydrophilic polymer having a molecular weight of 1,000 to 100,000 daltons, and is selected from the group consisting of: poly(ethylene glycol) (PEG), hyaluronic acid, dextran, polyethylenimine, poloxamers, povidone, D-alpha-tocopheryl, and polyethylene glycol succinate;
the flavonoid is EGCG, EC, EGC, or ECG, as shown in the structures below: 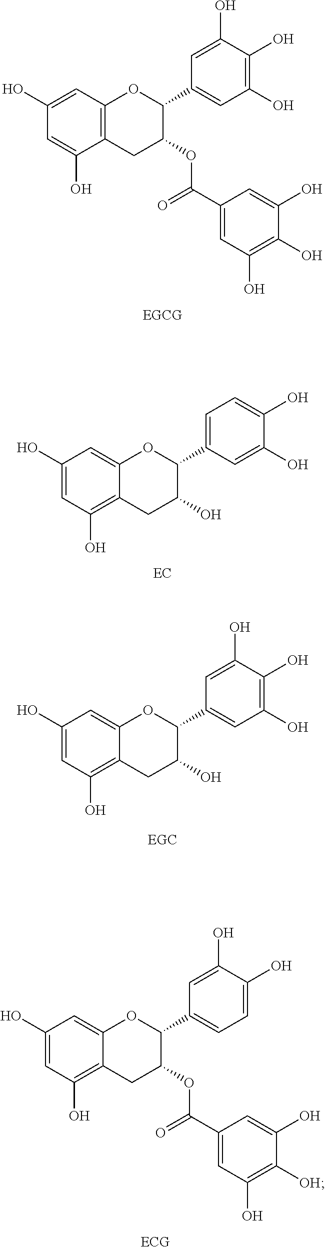 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
the flavonoid oligomer comprises 2-20 flavonoids of EGCG, EC, EGC, or ECG;
the infection is caused by bacteria or viruses selected from the group consisting of: severe acute respiratory syndrome coronavirus (SARS-CoV), enterovirus virus, HIV, hepatitis B virus, MERS-CoV, influenza virus, Dengue virus, respiratory syncytial virus, hepatitis C virus, monkeypox virus, human papillomavirus, methicillin-resistant Staphylococcus aureus, Pseudomonas, Bacillus anthracis, Tetani bacterium, Streptococcus pneumoniae, Meningococcus, Escherichia coli, Legionella, Neisseria gonorrhea, Neisseria meningitidis, and Salmonella.

2. The method according to claim 1, wherein the micelles having an outer shell comprising PEG-EGCG and an inner shell comprising EGCG oligomers.

3. The method according to claim 1, wherein the infection is caused by severe acute respiratory syndrome coronavirus 2, and the drug is IFN-α, IFN-β, IL-6, IL-12, IL-21, anti-IL-6, anti-IL-6R, Spike vaccine, RBD vaccine, ACE2 antagonist, anti-TMPRSS2, anti-CD147, anti-VEGFA, anti-GM-CSF, dexamethasone, chloroquine, Nsp12-RdRp inhibitor, hydroxychloroquine, anti-3CLpro, interferon-a, AAK-1 inhibitor, or JAK inhibitor.

4. The method according to claim 1, wherein the infection is caused by enterovirus virus, and the drug is IFN-α, IFN-β, Capsid binder, 3Cpro inhibitor, 3Dpol inhibitor, 2CATPase inhibitor, 2APro inhibitor, anti-HSP90, anti-PI4KB, anti-OSBP, anti-RdRP, SP40, SP45, SP55, SP81, LVLQTM, VAD, or AAPV.

5. The method according to claim 1, wherein the infection is caused by HIV, and the drug is IFN-α, anti-tetanus immunoglobulin, tetanus toxoid, benzodiazepines, magnesium sulfate, intrathecal baclofen, dantrolene, ketamine, propofol, botulinum toxin, human anti-tetanus immunoglobulin, or tetanus toxoid vaccine.

6. The method according to claim 1, wherein the infection is caused by hepatitis B virus, and the drug is IFN-α, IFN-γ, IL-12, IL-6, IL-21, DNA polymerase reverse transcriptase activity inhibitor, preS1 peptide, CRISPR/Cas9, ZFNs, capsid assembly modulator, E-neg and E-pos RNAi, Lamivudine, adefovir, entecavir, Tenofovir, TLR agonist, STING agonist, or cyclophilin inhibitor.

7. The method according to claim 1, wherein the infection is caused by MERS-CoV, and the drug is IFN-α, IFN-β, IFN-γ, anti-DPP4, Poly IC, anti-ACE2 antagonist, RdRp inhibitor, Chlorpromazine hydrochloride, Chloroquine, Peptide, endosome protease inhibitor, TMPRSS2 inhibitor, Furin protease inhibitor, anti-clathrin endocytosis, MERS-CoV S DNA, or RBD subunit vaccine.

8. The method according to claim 1, wherein the infection is caused by influenza virus, and the drug is IFN-α, IFN-β, IFN-γ, IL-12, IL-6, IL-15, IL-21, neuraminidase inhibitor, cap dependent endonuclease inhibitor, M2 ion channel blockers, nucleoprotein inhibitor, anti-NS1-1, anti-CPSF30, anti-PABII, anti-eIF4G1, anti-PABP1, anti-p85, anti-PKR, anti-PACT, anti-NXF1, anti-p15, anti-Importin, anti-crk, anti-crkL, anti-RIG-1, anti-nucleolin, anti-TRIM25, anti-Gas8, anti-Akt, anti-p53, antiPARP10, anti-RIL, anti-Hsp90, anti-PDZ, anti-NOLC1, anti-RAP55, anti-IKK, anti-hPAF1C, or anti-hGBP1.

9. The method according to claim 1, wherein the infection is caused by dengue virus, and the drug is anti-TNF-α, anti-IL-6, anti-RANTES, Chloroquine, Prednisolone, NS5 nucleoside inhibitor, ER-associated α, Glucosidase inhibitor, Lovastatin, capsid inhibitor, envelope inhibitor, anti-NS4B, anti-NS2B/3, anti-NS1, or NS1 vaccine.

10. The method according to claim 1, wherein the infection is caused by respiratory syncytial virus, and the drug is IL-15, anti-TNF-α, Benzimidazole derivatives, disulfonated stilbene, imidazoiso-indolone derivative, triphenolic compound, anti-envelope glycoproteins, sulfated sialyl lipid, anti-NS1, anti-F glycoprotein, siRNA inhibit P protein, NS1 protein, or N protein genes.

11. The method according to claim 1, wherein the infection is caused by SARS-CoV, and the drug is IFN-α, IFN-β, IL-21, anti-IL-6, anti-IL-6R, anti-VEGFA, anti-GM-CSF, Spike vaccine, RBD vaccine, ACE2 antagonist, anti-TMPRSS2, anti-CD147, dexamethasone, chloroquine, Nsp12-RdRp inhibitor, hydroxychloroquine, anti-3CLpro, Interferon-α, AAK-1inhibitor, JAK inhibitor, anti-ATM, anti-SIRT1, anti-GSK3B, or anti-Torin-2.

12. The method according to claim 1, wherein the infection is caused by hepatitis C virus, and the drug is IFN-α, IFN-β, IL-21, anti-IL-6, anti-IL-6R, Anti-EGFR, Anti-NS2, Anti-NS3, Anti-NS4A, Anti-NS5A, Anti-NS5B, Anti-Helicase, Anti-TLR-9, Anti-HCV E2, Anti-E1, Anti-E2, Anti-p7, Anti-CD81, Anti-SRB1, Anti-CLDN1, Anti-EphA2, Anti-TfR1, Anti-NPC1L1, Cyclosporin A, Anti-Alpha-glucosidase, Anti-DGAT-1, Anti-VLDL, Anti-CD81, Anti-CLDN1 or Anti-SR-B1.

13. A method of treating a bacterial or viral infection, comprising the step of administering to a subject in need thereof an effective amount of a polymer-flavonoid conjugate,
wherein the polymer is a hydrophilic polymer having a molecular weight of 1,000 to 100,000 daltons, and is selected from the group consisting of: PEG, hyaluronic acid, dextran, polyethylenimine, poloxamers, povidone, D-alpha-tocopheryl, and polyethylene glycol succinate;
the flavonoid is EGCG, EC, EGC, or ECG, as shown in the structures below: 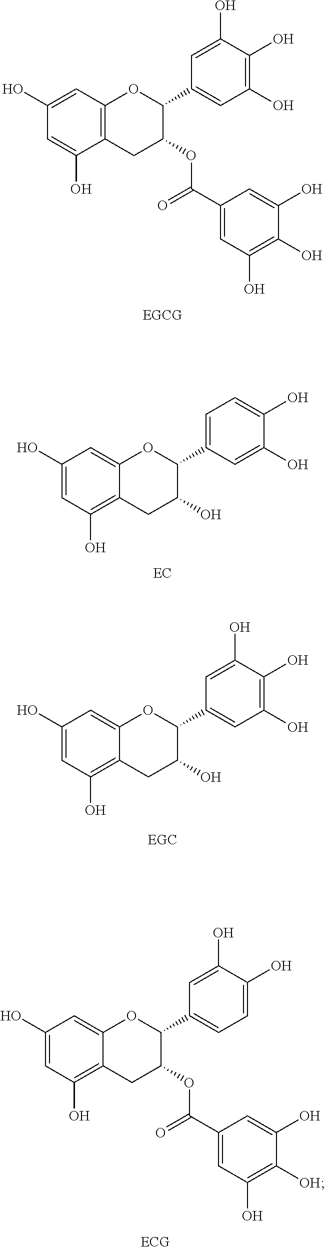 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
and
the infection is caused by DNA viruses, RNA viruses, gram-positive bacteria, or gram-negative bacteria.

14. The method according to claim 13, wherein the polymer-flavonoid conjugate is PEG-EGCG.

15. The method according to claim 13, wherein the infection is caused by a DNA virus selected from the group consisting of: hepatitis B virus, human herpesvirus, human papillomavirus, herpes simplex virus, Epstein-Barr virus, cytomegalovirus, monkeypox virus and Varicella-zoster virus.

16. The method according to claim 13, wherein the infection is caused by an RNA virus selected from the group consisting of: SARS-CoV-2, enterovirus virus, HIV, MERS-CoV, hepatitis C virus, hepatitis A virus, rotavirus, norovirus, influenza virus, parainfluenza virus, Dengue virus, respiratory syncytial virus, and SARS-CoV.

17. A method of treating a bacterial or viral infection, comprising the step of administering to a subject in need thereof an effective amount of a flavonoid oligomer,
wherein the flavonoid is EGCG, EC, EGC, or ECG, as shown in the structures below: 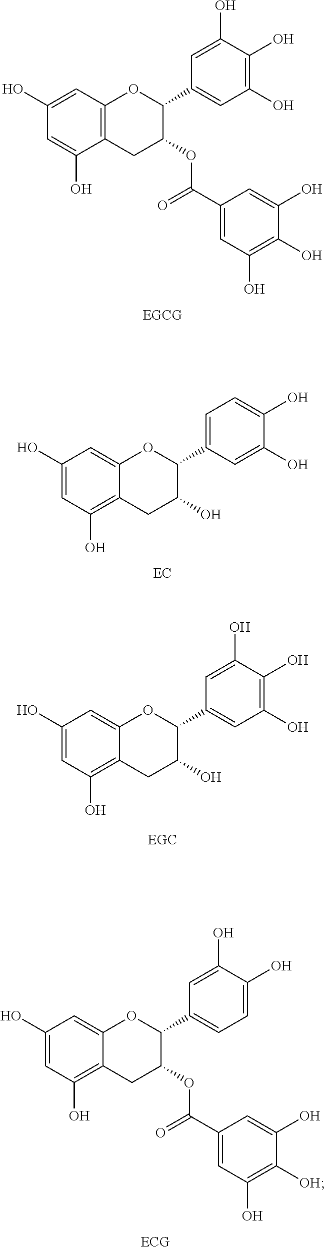 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
the flavonoid oligomer comprises 4-12 flavonoids of EGCG, EC, EGC, or ECG;
the infection is caused by DNA viruses, RNA viruses, gram-positive bacteria, or gram-negative bacteria.

18. The method according to claim 17, wherein the flavonoid oligomer is oligomer of EGCG.

19. The method according to claim 17, wherein the infection is caused by a DNA virus selected from the group consisting of: hepatitis B virus, human herpesvirus, human papillomavirus, herpes simplex virus, Epstein-Barr virus, cytomegalovirus, monkeypox virus and Varicella-zoster virus.

20. The method according to claim 17, wherein the infection is caused by an RNA virus selected from the group consisting of: SARS-CoV-2, enterovirus virus, HIV, MERS-CoV, hepatitis C virus, hepatitis A virus, rotavirus, norovirus, influenza virus, parainfluenza virus, Dengue virus, respiratory syncytial virus, and SARS-CoV.
